(19) United States
(12) Patent Application Publication
(10) Pub. No.: US 2024/0050491 A1
(43) Pub. Date: Feb. 15, 2024

(54) ANTIMICROBIAL AGENTS DERIVED FROM BACILLUS

(71) Applicant: The state of Israel, Ministry of Agriculture & Rural Development, Agricultural Research Organization, Rishon-LeZion (IL)

(72) Inventors: Moshe SHEMESH, ModiIn (IL); Yulia KROUPITSKI, Ness Ziona (IL); Shlomo SELA, ModiIn (IL)

(73) Assignee: The state of Israel, Ministry of Agriculture & Rural Development, Agricultural Research Organization, Rishon-LeZion (IL)

(21) Appl. No.: 18/368,040

(22) Filed: Sep. 14, 2023

Related U.S. Application Data
(63) Continuation of application No. PCT/IL2022/050289, filed on Mar. 14, 2022.
(60) Provisional application No. 63/160,829, filed on Mar. 14, 2021.
(60) Provisional application No. 63/184,264, filed on May 5, 2021.

(51) Int. Cl. A61K 35/742; A01N 3/00; A61P 31/04; A01N 63/22; C12N 1/20; A23L 3/3463; A23L 3/3571; A23B 7/155

(52) U.S. Cl. CPC A61K 35/742; A01N 3/00; A61P 31/04; A01N 63/22; C12N 1/20; A23L 3/34635; A23L 3/3571; A23B 7/155

(57) ABSTRACT

A method of generating an antimicrobial agent is disclosed. The method comprises:

(a) culturing Bacillus cells in a medium under conditions effective to allow secretion of at least one antimicrobial agent into the medium; and

(b) purifying the at least one antimicrobial agent from the medium to generate a purified preparation comprising the at least one antimicrobial agent.

Uses of the antimicrobial agents secreted from the Bacillus cells are also disclosed.

### RELATED APPLICATIONS

This application is a Continuation of PCT Patent Application No. PCT/IL2022/050289 having International filing date of Mar. 14, 2022, which claims the benefit of priority under 35 USC § 119(e) of U.S. Provisional Patent Application Nos. 63/160,829 filed on Mar. 14, 2021 and 63/184,264 filed on May 5, 2021. The contents of the above applications are all incorporated by reference as if fully set forth herein in their entirety.

### SEQUENCE LISTING STATEMENT

The XML file, entitled 97829 SequenceListing.xml, created on Sep. 12, 2023, comprising 8,393,361 bytes, submitted concurrently with the filing of this application is incorporated herein by reference.

### FIELD AND BACKGROUND OF THE INVENTION

The present invention, in some embodiments thereof, relates to antimicrobial agents derived from Bacillus species and, more particularly, but not exclusively, to antibacterial agents derived from Bacillus subtilis.

Non-typhoidal Salmonella enterica serovars are of major public health concern, leading to approximately 93.8 million cases of gastroenteritis and 155,000 deaths each year. In the United States and worldwide, Salmonella is the leading bacterial pathogen responsible for food-borne outbreaks. Efforts to control Salmonella in food include improved hygienic methods, vaccination, probiotics, prebiotics and use of antimicrobial agents. However, the rise of multidrug antibiotic 30 resistance has increased pressure to reduce the use of a wide range antimicrobial agents, including antibiotics in the food production. Since, outbreaks of Salmonellosis due to egg or meat contamination still occur, it is of high priority to develop efficient means to interfere with the pathogen's transmission through the production chain.

Bacillus subtilis represents a potentially biocontrol bacterium against pathogenic species such as Salmonella enterica. Beside their spore-forming ability, Bacillus species are capable of surviving stressful environments due to their capacity to produce complex communities of multicellular cells e.g. biofilms. Cells in biofilms are bound together by extracellular polymorphic substances (EPS) and form complex structures, capable of resisting environmental stresses including antimicrobial agents. Thus, biofilm formation represents a strategy for persistence under unfavorable conditions in diverse environments, such as competition among different microorganisms.

Consumption of sprouts is increasing in the world as they are a good source of various nutrients that include proteins, carbohydrates, minerals, and Sprout seeds are germinated under warm and humid conditions that are ideal and optimal for bacterial proliferation. The germination step is the main source of contamination in sprouts as bacteria present in the seeds may become internalized during the sprouting. These bacteria include human pathogens bacteria such as Salmonella enterica and E. coli. Sprouts are usually consumed in the raw form in western countries so that the nutrients and other components that confer health benefits are not lost. Hence, there is a high risk involved in sprout consumption because in most cases there are no steps taken before consumption to eliminate the pathogenic bacteria that may be present. In recent decades, foodborne disease outbreaks due to the consumption of sprouts have occurred worldwide. The bacterial pathogens responsible for these outbreaks are largely Salmonella spp. and pathogenic Escherichia coli strains O157:H7 and 0104. Foodborne illness caused by raw sprout consumptions is mainly associated with alfalfa sprouts, likely because alfalfa is among the most widely available crops. The contamination of sprouts has become a worldwide food safety concern. The focus on prevention based strategies becomes very important. Intervention methods aimed at reducing or eliminating bacterial populations following pre-harvest and post-harvest contamination in sprouts have been studied. These methods include physical, biological, as well as chemical interventions.

These methods have been explored to reduce foodborne pathogens on sprouts and seeds, although their effectiveness has been suboptimal.

Chlorine is the most commonly used sanitizing agent for treatment of fresh produce products in the United States. The U.S. Food and Drug Administration (FDA) recommends sanitizing seeds with 20,000 ppm active chlorine, as calcium hypochlorite (Ca(OCl)₂), for 15 min, and to monitor the bacteria count in spent irrigation water as a means to control risk of foodborne illness associated with sprouts. Some promising results have been reported for chlorine treatment in eliminating pathogens on sprouts and their seeds with no significant negative impact on seed germination capacity. However, most studies have reported that chlorine is ineffective in the complete eradication of pathogens from vegetable surfaces, particularly if the initial bacterial load is high. The average microbial reduction by chlorine treatment was reported to be about 2.5 log CFU/g, which is much lower than the 5-log reduction recommended by the National Advisory Committee on Microbiological Criteria for Foods.

The strategies for food bio-preservation are generally based on the use of antagonistic microorganisms, antimicrobial metabolites, and bacteriophages. Nonpathogenic microorganisms that can compete with pathogens for physical space and nutrients have been applied to reduce foodborne pathogens in food products.

Over the last decades, applications using members of the Bacillus subtilis group have emerged in both food processes and crop protection industries. The B. subtilis group offers an abundance of antagonistic compounds displaying a broad range of biological functions. This huge versatility increases the industrial and environmental interest of B. subtilis strains, especially when considering their range of action against foodborne or phytopathogenic flora as well as their history of safe use in food.

Background art includes Shemesh and Chai, J Bacteriol. 2013 June; 195(12): 2747-2754. Borriss, R. 2011 Use of plant-associated Bacillus strains as biofertilizers and biocontrol agents, In: Maheshwari DK (ed). Bacteria in agrobiology: plant growth responses. Springer. Germany, pp 41-76; Dhouib, H., Zouari, I., Abdallah, D. B., Belbahri, L, Taktak, W, Triki, M. A., & Tounsi. S. 2019. Potential of a novel endophytic Bacillus velezensis in tomato growth promotion and protection against Verticillin wilt disease. Biological Control, 139, 104092; and Fan B, Wang C, Song X, Ding X, Wu L, Wu H, Gao X, Borriss R. 2018. Bacillus velezensis FZB42 in 2018: The gram-positive model strain for plant growth promotion and biocontrol. Front Microbiol 9:1-14.

### SUMMARY OF THE INVENTION

According to an aspect of the present invention there is provided a method of generating an antimicrobial agent comprising:
(a) culturing Bacillus cells of the species subtilis or velezensis in a medium under conditions effective to allow secretion of at least one antimicrobial agent into the medium; and
(b) purifying said at least one antimicrobial agent from the medium to generate a purified preparation comprising said at least one antimicrobial agent, thereby generating the antimicrobial agent.

According to an aspect of the present invention there is provided a composition of matter comprising at least one antimicrobial agent generated according to the method described herein.

According to an aspect of the present invention there is provided a composition of matter comprising at least one antimicrobial agent secreted from a Bacillus bacteria of the species subtilis or velezensis, wherein said at least one antimicrobial agent is more than 1% of the Bacillus components in the composition of matter.

According to an aspect of the present invention there is provided a method of treating an infection in a subject in need thereof, the method comprising administering to the subject a therapeutically effective amount of the composition of matter described herein, thereby treating the infection.

According to an aspect of the present invention there is provided a solid support coated with at least one antimicrobial agent generated according to the method described herein or the composition described herein.

According to an aspect of the present invention there is provided a method of killing a microbe, the method comprising contacting the microbe with at least one antimicrobial agent generated according to the method described herein or the composition described herein, thereby killing the microbe.

According to an aspect of the present invention there is provided a bacterial culture comprising Bacillus bacteria of the species subtilis or velezensis and a heterologous plant which promotes antimicrobial activity of said Bacillus bacteria.

According to an aspect of the present invention there is provided a conditioned medium of Bacillus bacteria having a 16S rRNA sequence as set forth in SEQ ID NO: 1 or 3.

According to an aspect of the present invention there is provided an article of manufacture comprising Bacillus bacteria having a 16S rRNA sequence as set forth in SEQ ID NO: 1 or 3 and an agriculturally acceptable carrier.

According to an aspect of the present invention there is provided a method for prolonging the shelf life of a plant or the post-harvest quality of a plant, or a combination thereof comprising post-harvest contacting said plant with an effective amount of Bacillus bacteria of the species subtilis or velezensis, thereby prolonging the shelf life of a plant or the post-harvest quality of a plant, or a combination thereof.

According to an aspect of the present invention there is provided a method for prolonging the shelf life of a plant, the post-harvest quality of a plant, increasing a plant performance parameter, or a combination thereof comprising pre-harvest contacting said plant with an effective amount of Bacillus bacteria having a 16S rRNA sequence as set forth in SEQ ID NOs: 1 or 3, thereby prolonging the shelf life of a plant, the post-harvest quality of a plant, or a combination thereof.

According to an aspect of the present invention there is provided a method of preserving a food product comprising contacting the food with a Bacillus bacteria of the species Subtilis or Velezensis or an agent derived from said Bacillus bacteria under conditions that down-regulate an activity and/or an amount of at least one type of microbe that is capable of spoiling the food product, thereby preserving the food product.

According to embodiments of the present invention, the at least one antimicrobial agent comprises more than 20% of the total Bacillus components in the preparation.

According to embodiments of the present invention, the method further comprises testing the activity of the antimicrobial agent.

According to embodiments of the present invention, the Bacillus cells are of the species subtilis.

According to embodiments of the present invention, the conditions support generation of a biofilm of said Bacillus subtilis cells.

According to embodiments of the present invention, the conditions enhance secretion of a pigment secreted from said Bacillus subtilis cells.

According to embodiments of the present invention, the purifying is effected by size exclusion.

According to embodiments of the present invention, the purifying is effected using reverse phase column chromatography.

According to embodiments of the present invention, the antimicrobial agent is an antibacterial agent.

According to embodiments of the present invention, the antibacterial agent is bactericidal towards at least one Gram positive bacteria.

According to embodiments of the present invention, the antibacterial agent is bactericidal towards at least one Gram negative bacteria.

According to embodiments of the present invention, the antibacterial agent is bactericidal towards at least one of the bacteria selected from the group consisting of E. coli, Salmonella Enterica, Staphylococcus aureus, Staphylococcus epidermidis and Bacillus cereus.

According to embodiments of the present invention, the Bacillus velezensis has a 16S rRNA sequence as set forth in SEQ ID NO: 1.

According to embodiments of the present invention, the composition further comprises a pharmaceutically acceptable carrier.

According to embodiments of the present invention, the infection is a bacterial infection.

According to embodiments of the present invention, the bacterial infection is selected from the group consisting of an E. coli infection, a Salmonella Enterica infection, a Staphylococcus aureus infection, a Staphylococcus epidermidis infection and a Bacillus cereus infection.

According to embodiments of the present invention, the contacting is effected in vivo.

According to embodiments of the present invention, the contacting is effected ex vivo.

According to embodiments of the present invention, the microbe comprises a bacteria.

According to embodiments of the present invention, the microbe is present on a plant or in the vicinity thereof.

According to embodiments of the present invention, the plant is a sprout.

According to embodiments of the present invention, the sprout is a seedling.

According to embodiments of the present invention, the sprout is a Medicago sativa sprout or a Vigna radiate sprout.

According to embodiments of the present invention, the bacteria of the bacterial culture has a 16S rRNA sequence as set forth in SEQ ID NO: 1.

According to embodiments of the present invention, the bacterial culture According to embodiments of the present invention, the further comprises an agriculturally acceptable carrier.

According to embodiments of the present invention, more than 90% of the bacteria of the bacterial culture is the Bacillus bacteria.

According to embodiments of the present invention, the agriculturally acceptable carrier comprises at least one agent selected from the group consisting of a stabilizer, a tackifier, a preservative, a carrier, a surfactant, an anticomplex agent and a combination thereof.

According to embodiments of the present invention, the bacterial culture is lyophilized.

According to embodiments of the present invention, the article of manufacture further comprises an agent which promotes the growth of a plant.

According to embodiments of the present invention, the agent which promotes the growth of the plant is selected from the group consisting of a fertilizer, an acaricide, a heribicide, a fungicide, an insecticide, a nematicide, a pesticide, a plant growth regulator, a rodenticide and a nutrient.

According to embodiments of the present invention, the plant is a crop plant.

According to embodiments of the present invention, the crop plant is a cultivated crop plant.

According to embodiments of the present invention, the plant is a monocot.

According to embodiments of the present invention, the plant is a dicot.

According to embodiments of the present invention, the Bacillus bacteria has a 16S rRNA sequence as set forth in SEQ ID NO: 1.

According to embodiments of the present invention, the Bacillus bacteria are formulated in a composition selected from the group consisting of: a dip, a spray, a seed coating and a concentrate.

According to embodiments of the present invention, the plant is at: a post-blossom stage, a blossom stage, a pre-blossom stage, or any combination thereof.

According to embodiments of the present invention, the contacting is contacting in the vicinity of or onto: a root, a stem, a trunk, a seed, a fruit, a flower, a leaf, or any combination thereof.

According to embodiments of the present invention, the food product is a fruit or vegetable.

According to embodiments of the present invention, the Bacillus bacteria has a 16S rRNA sequence as set forth in SEQ ID NO: 1 or 3.

According to embodiments of the present invention, the method further comprises contacting the plant with chlorine.

According to embodiments of the present invention, the method further comprises contacting the food with chlorine.

According to embodiments of the present invention, the microbe is a bacteria.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B are photographs of Bacillus subtilis 3610 colony morphology on LB plus 1% [vol/vol] glycerol and 0.1 mM MnSO₄ (LBGM), Liquid microbial growth medium (LB) and Nutrient Agar (NA) 72 hours (FIG. 1A) and Salmonella inhibition zone around Bacillus colony slab (FIG. 1B).

FIGS. 2A-B are photographs of Salmonella and Bacillus colonies cultured on LBGM (A) or LB (B) agar.

FIGS. 3A-B are photographs illustrating Bacillus colony formation on LBGM agar at 24, 48 and 72 hours (FIG. 3A) and the Salmonella inhibition zone around Bacillus colony slab (FIG. 3B).

FIGS. 4A-B are photographs illustrating colony morphology of 3610 wt and biofilm mutants on LBGM agar (FIG. 4A) and the Salmonella inhibition zone around Bacillus colony slab (FIG. 4B).

FIG. 5 is a photograph of the Staphylococcus aureus inhibition zone.

FIG. 6 is a photograph illustrating the anti-microbial activity of the brown and the black pigments derived from Bacillus subtilis in the plate model. Zone of inhibition (diameter in mm) was measured around the pigment well.

FIGS. 7A-B are graphs illustrating the antimicrobial effect of the purified pigment during growth of bacterial suspension. The tested pathogens at 10⁴ colony forming unit (CFU)/ml concentration were exposed to the purified pigment during their growth for 24 h at 25° C. The experiment was performed in triplicate.

FIGS. 8A-B: Salmonella numbers presented in log CFU/ml after 24 h, 48 h, and 72 h incubation in NB and boiled and filtrated sprout medium (BFSM). Salmonella was incubated in NB or BASF with or without Bacillus velezensis (strain BX77) cells at an initial concentration of 10² cells/ml. Initial concentration of Bacillus was either 10² CFU/ml (low load) or 10⁷ CFU/ml (high load). Black asterisks demonstrate total killing of Salmonella cells.

FIG. 9 is a photograph of the BFSA (boiled filtrated sprout agar) which becomes yellow/brown in the presence of Bacillus cells within 72 hours.

FIG. 10 is a graph presenting Salmonella numbers in log CFU/ml after 24 h in non-filtrated BFSA and BFSA filtrated from BX77. Salmonella was incubated in BFSA at an initial concentration of 10² CFU/ml or 10⁷ CFU/ml. Black asterisk showed total killing of Salmonella cells. This experiment was performed 4 times.

FIGS. 11A-B illustrate the antimicrobial activity of Bacillus cells against Salmonella on alfalfa roots. Red fluorescence of Salmonella cells and green fluorescence of Bacillus subtilis cells were visualized on alfalfa root by Confocal laser scanning microscopy. FIG. 11A demonstrates the colonization of the root with Salmonella, while FIG. 11B image shows an eradication of Salmonella in presence of Bacillus cells.

FIG. 12 is a photograph illustrating the inhibition of Salmonella (horizontal lines) by BX77 and B. subtilis 3610 (vertical lines) on LB (left panel) and plant extract agar BFSA (right panel). Inhibition zone is seen in the cross-section of the lines.

FIG. 13A is a graph illustrating the kinetics of Salmonella inactivation in conditioned-medium prepared from BX77 culture in Medicago sativa extract (also referred to as BFSA).

Salmonella (10⁴ CFU/ml) was added to a fresh, sterile plant's extract (SE; blue) or to a conditioned medium (CM; orange) prepared following the growth of BX77 in the plant's extract for 72 h at 25° C.

FIG. 13B is a graph illustrating the inhibition of Salmonella growth by conditioned-medium prepared from 72 h culture of strain BX77 in Medicago saliva extract (CM-BX77), or in Vigna radiate extract (MCM-BX77). Salmonella (10⁴ CFU/ml) was incubated in the plants extracts for 24 h at 25° C. BF, Medicago saliva extract; MBF, Vigna radiate extract.

FIGS. 14A-C are graphs and photographs illustrating the relationship between Bacillus BX77 growth, sporulation and generation of antimicrobial agents. BX77 was grown in plant extract (BF), or LB broth for 72 h at 25° C. Sporulation was assessed microscopically at different time points and is presented as percent sporulation relative to the entire Bacillus population (A). The activity of CM-samples, taken at the same time points, against Salmonella was determined by adding Salmonella (10⁴ CFU/ml) and incubating for another 24 h at 25° C. (B). A bright-field image of the cultures at 48 h is shown in FIG. 14C, indicating the high abundance of endospores (white dots) in the BF relative to the LB culture.

FIG. 15 is a photograph illustrating the antifungal activity of BX77. Fungi were grown on nutrient agar alone (top panel), or in the presence of Bacillus BX77 streaked as a line in the middle of each plate (Bottom panel). A, Aspergillus flavus; B, Fusarium proliferatum; C, Pencillium expansum.

FIG. 16 are photographs illustrating that Bacillus BX77 inhibits the colonization of Salmonella on 4-days old sprouted Medicago saliva seeds.

FIG. 17 is a graph illustrating the expression kinetics of Bacillus BX77 genes involved in the synthesis of known antimicrobial agents.

FIGS. 18A-B are graphs illustrating the influence of decontamination by Ca-hypochlorite, of Salmonella contaminated alfalfa seeds, on the concentration of Salmonella. The seeds were contaminated with Salmonella by dipping in 10²-109 CFU/ml. Decontamination was performed with 20,000 ppm Ca-hypochlorite. The number of Salmonella on the seeds (CFU/g seeds) was detected immediately after the contamination (A), and in the sprouts (B) after 3 days of sprouting (CFU/g sprouts). The results are representing the average and standard error of 3 independent experiments in duplicates. When bacteria were not detected, enrichment of the samples was performed in BPW. (*) Growth after enrichment. (−) No growth.

FIG. 19 is a graph illustrating Salmonella Typhimurium SL1344 survival in sprouts in the presence of BX-77 or absence of BX-77 after disinfection by Ca-hypochlorite of Salmonella contaminated seeds. Alfalfa seeds were contaminated by 10² or 10⁵ CFU of Salmonella, dried, decontaminated by Ca-hypochlorite and seeded in the presence or absence of BX-77. Salmonella infected seeds were used as controls in the absence or the presence of BX-77. The experiment was repeated twice and each set included 5 cups.

### DESCRIPTION OF SPECIFIC EMBODIMENTS OF THE INVENTION

The present invention, in some embodiments thereof, relates to antimicrobial agents derived from Bacillus species and, more particularly, but not exclusively, to antibacterial agents derived from Bacillus subtilis or Bacillus velezensis.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Bacillus subtilis and Bacillus velezensis represent potentially biocontrol bacteria against pathogenic species such as Salmonella. Beside their spore-forming ability, Bacillus species are capable of surviving stressful environments due to their capacity to produce complex communities of multicellular cells, such as biofilms. Cells in biofilms are bound together by extracellular polymorphic substances (EPS) and form complex structures, capable of resisting environmental stresses including antimicrobial agents. Thus, biofilm formation represents a strategy for persistence under unfavorable conditions in diverse environments, such as competition among different microorganisms.

A recent study demonstrated the ability of B. subtilis in production a dark brown pigment during biofilm formation in LBGM medium (Shemesh and Chai, J Bacteriol. 2013 June; 195(12): 2747-2754). It was noted that this pigment production was associated with the ability of B. subtilis to form a robust colony-type biofilm.

The present inventors hypothesized that the pigment produced by B. subtilis during biofilm formation may comprise antimicrobial properties enabling this bacterium to compete with different microbial species.

Whilst reducing the present invention to practice, the present inventors have shown that agents secreted from the biofilm generated from Bacillus subtilis were bactericidal towards a variety of bacteria including E. coli, Salmonella Enterica, Staphylococcus aureus, Staphylococcus epidermidis and Bacillus cereus, as summarized in Table 2 of the Examples section herein below.

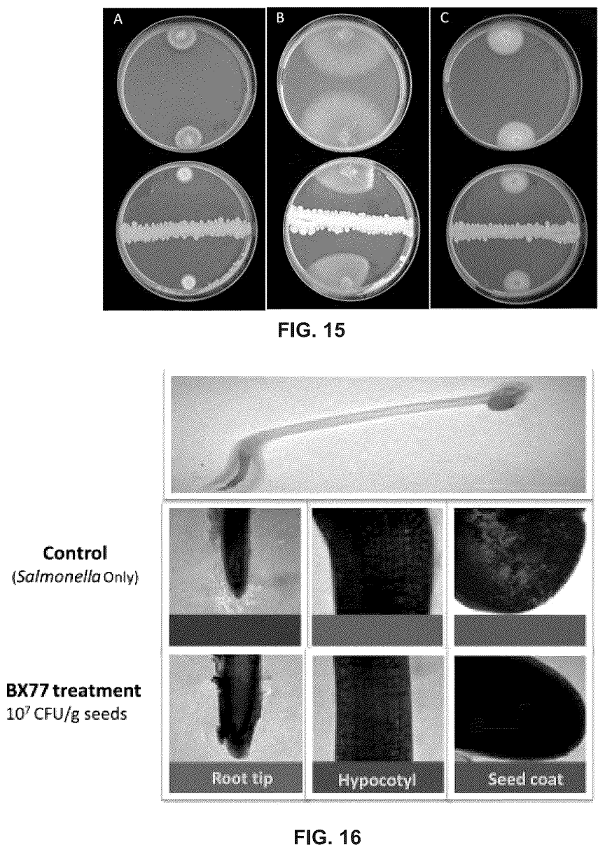

Whilst further reducing the present invention to practice, the present inventors have shown that the Bacillus species (referred to herein as BX77) has very strong antimicrobial activities towards several bacteria and fungi (FIG. 15).

The present inventors propose that this bacteria may be useful at management of crops at both the pre—and post-harvest stage. More specifically, the present inventors envisage that secreted antimicrobials from these bacteria may be useful as a food bio-preservative.

Thus, according to a first aspect of the present invention, there is provided a method of generating an antimicrobial agent comprising:
(a) culturing Bacillus cells of the species subtilis or velezensis in a medium under conditions effective to allow secretion of at least one antimicrobial agent into the medium; and
(b) purifying the at least one antimicrobial agent from the medium to generate a purified preparation comprising the at least one antimicrobial agent, thereby generating the antimicrobial agent.

As used herein, the term “antimicrobial agent” refers to an agent having antimicrobial activity—i.e. the ability to suppress, control, inhibit or kill microorganisms, such as bacteria, fungi, viruses, protists and archae.

According to a particular embodiment, the antimicrobial agent is not an antifungal agent.

According to a specific embodiment, the antimicrobial agent is an antibacterial agent.

According to a specific embodiment, the antimicrobial agent is devoid of antiviral activity.

The antibacterial agent of this aspect of the present invention may comprise bactericidal and/or bacteriostatic towards at least one gram positive bacteria and/or one gram negative bacteria.

The term “Gram-positive bacteria” as used herein refers to bacteria characterized by having as part of their cell wall structure peptidoglycan as well as polysaccharides and/or teichoic acids and are characterized by their blue-violet color reaction in the Gram-staining procedure.

Representative Gram-positive bacteria include: Actinomyces spp., Bacillus anthracis, Bifidobacterium spp., Clostridium botulinum, Clostridium perfringens, Clostridium spp., Clostridium tetani, Corynebacterium diphtheriae, Corynebacterium jeikeium, Enterococcus faecalis, Enterococcus faecium, Erysipelothrix rhusiopathiae, Eubacterium spp., Gardnerella vaginalis, Gemella morbillorum, Leuconostoc spp., Mycobacterium abcessus, Mycobacterium avium complex, Mycobacterium chelonae, Mycobacterium fortuitum, Mycobacterium haemophilium, Mycobacterium kansasii, Mycobacterium leprae, Mycobacterium marinum, Mycobacterium scrofulaceum, Mycobacterium smegmatis, Mycobacterium terrae, Mycobacterium tuberculosis, Mycobacterium ulcerans, Nocardia spp., Peptococcus niger, Peptostreptococcus spp., Proprionibacterium spp., Staphylococcus aureus, Staphylococcus auricularis, Staphylococcus capitis, Staphylococcus cohnii, Staphylococcus epidermidis, Staphylococcus haemolyticus, Staphylococcus hominis, Staphylococcus lugdanensis, Staphylococcus saccharolyticus, Staphylococcus saprophyticus, Staphylococcus schleiferi, Staphylococcus similans, Staphylococcus warneri, Staphylococcus xylosus, Streptococcus agalactiae (group B streptococcus), Streptococcus anginosus, Streptococcus bovis, Streptococcus canis, Streptococcus equi, Streptococcus milleri, Streptococcus mitior, Streptococcus mutans, Streptococcus pneumoniae, Streptococcus pyogenes (group A streptococcus), Streptococcus salivarius, Streptococcus sanguis.

The term “Gram-negative bacteria” as used herein refer to bacteria characterized by the presence of a double membrane surrounding each bacterial cell. Representative Gram-negative bacteria include Acinetobacter calcoaceticus, Actinobacillus actinomycetemcomitans, Aeromonas hydrophila, Alcaligenes xylosoxidans, Bacteroides, Bacteroides fragilis, Bartonella bacilliformis, Bordetella spp., Borrelia burgdorferi, Branhamella catarrhalis, Brucella spp., Campylobacter spp., Chalmydia pneumoniae, Chlamydia psittaci, Chlamydia trachomatis, Chromobacterium violaceum, Citrobacter spp., Eikenella corrodens, Enterobacter aerogenes, Escherichia coli, Flavobacterium meningosepticum, Fusobacterium spp., Haemophilus influenzae, Haemophilus spp., Helicobacter pylori, Klebsiella spp., Legionella spp., Leptospira spp., Moraxella catarrhalis, Morganella morganii, Mycoplasma pneumoniae, Neisseria gonorrhoeae, Neisseria meningitidis, Pasteurella multocida, Plesiomonas shigelloides, Prevotella spp., Proteus spp., Providencia rettgeri, Pseudomonas aeruginosa, Pseudomonas spp., Rickettsia prowazekii, Rickettsia rickettsii, Rochalimaea spp., Salmonella spp., Salmonella typhi, Serratia marcescens, Shigella spp., Treponema carateum, Treponema pallidum, Treponema pallidum endemicum, Treponema pertenue, Veillonella spp., Vibrio cholerae, Vibrio vulnificus, Yersinia enterocolitica, Yersinia pestis.

According to particular embodiments, the agent of this aspect of the present invention comprises antibacterial activity against E. coli, Salmonella Enterica, Staphylococcus aureus, Staphylococcus epidermidis and Bacillus cereus.

According to another embodiment, the agent of this aspect of the present invention does not comprise antibacterial activity against a strain of Pseudomonas aeruginosa.

According to another embodiment, the agent of this aspect of the present invention has at least 1.5 times, 2 times and even 5 times greater antibacterial activity towards Staphylococcus aureus than towards Bacillus cereus, Salmonella Enterica, Pseudomonas aeruginosa or Enteropathogenic Escherichia coli, when measured in an identical assay under the same conditions.

As mentioned, in order to generate a purified preparation of the antimicrobial agent of this aspect of the present invention, the B. subtilis or B. velezensis bacteria are cultured in a medium under conditions effective to allow secretion of the antimicrobial agent into (or onto) the medium.

In one embodiment, the medium is a liquid medium (complex or minimal).

In another embodiment, the medium is a solid medium.

Exemplary strains of B. subtilis contemplated by the present invention include, but are not limited to B. subtilis NCIB 3610 (having a full genome sequence as set forth in SEQ ID NO: 2), Bacillus isolate BB124 (having a 16S rRNA sequence as set forth in SEQ ID NO: 3), B. subtilis MS1577 and 127185/2 (MS302; dairy isolate) and NCIB3610.

An exemplary strain of B. velezensis contemplated by the present invention include is BX77 (having a 16S rRNA sequence as set forth in SEQ ID NO: 1, and a full genome sequence as set forth in SEQ ID NO: 20).

Examples of growth substrates that can be used to culture Bacillus bacteria include but are not limited to MRS medium, LB medium, LBGS medium, TBS medium, yeast extract, soy peptone, casein peptone, milk and meat peptone.

Further examples of media are listed in Table 1 herein below.

TABLE 1
Abiotrophia media—Recipe for medium appropriate for growth of Abiotrophia genus
Acetamide Medium—Recipe for Acetamide medium.
Acetobacter Medium—Recipe for medium appropriate for the growth of Acetobacter genus.
Actinoplanes Medium—Media used to grow certain Actinoplanes species
Agrobacterium Agar Recipe—Agar appropriate for growth of Agrobacterium genus
Alicyclobacillus Agar—Recipe for Alicyclobacillus Agar.
Alicyclobacillus Medium—Recipe for Alicyclobacillus Medium.
Allantoin mineral agar—Recipe for the preparation of Allantoin minimal agar.
Allantoin mineral medium—Recipe for the preparation of Allantoin minimal medium.
Ashbya Full Medium—Recipe for the production of Ashbya full medium.
Azotobacter Agar—Agar appropriate for growth of Azobacter genus.
Bennett's Medium—media used for growth of some Actinoplanes species.
Bacillus agar—Agar used to grow some Bacillus species.
Bacillus broth—Agar used to grow some Bacillus species.
Bacillus schlegelii Medium—Medium appropriate for the growth of Bacillus schlegelii.
Bifidobacterium Medium—Recipe for Bifidobacterium medium.
Blue green algae agar—Recipe for blue green algae agar.
Blue green algae broth—Recipe for blue green algae broth.
Brain Heart Infusion Glucose Agar—Recipe for Brain Heart Infusion Glucose Agar.
Caulobacter Agar—Recipe for Caulobacter species
Caulobacter Medium—Recipe for Caulobacter spp.
Cantharellus Agar Recipe—Recipe for Cantharellus agar.
CASO agar—Recipe for CASO agar.
Clostridium thermocellum Medium—Recipe for medium appropriate for growth of
Clostridium thermocellum
Corynebacterium agar—Recipe for Corynebacterium agar.
Creatinine Medium—Recipe for the production of creatinine medium.
Czapek Agar (CZA)—Recipe for Czapek Agar (CZA).
Desulfovibrio Medium—Recipe for Desulfovibrio Medium.
Gluconobacter agar—Recipe for Gluconobacter agar.
Glucose Peptone Yeast Extract Agar (GPYA)—Recipe for Glucose Peptone Yeast Extract
Agar (GPYA).
Glucose Yeast Extract Agar—Recipe for Glucose Yeast Extract Agar.
Halobacterium agar—Recipe for the preparation of Halobacterium agar.
Halobacteria Medium—Recipe for Halobacteria Medium.
LB Agar—Recipe for the preparation of LB agar bacterial media.
LB broth—Recipe for the preparation of LB broth bacterial media.
LB broth (low salt)—Recipe for the preparation of low salt LB broth bacterial media.
Luminous Medium—Recipe for Luminous Medium.
M17 media—Recipe for the preparation of M17 media.
M9 minimal media—Minimal salts bacterial media.
Mannitol agar—Recipe for mannitol agar.
Mannitol broth—Recipe for mannitol broth.
Marine agar—Recipe for marine agar. Used for the growth of several marine bacteria.
Marine broth—Recipe for marine broth. Used for the growth of several marine bacteria.
Methylamine Salts Agar—Recipe for methylamine salts agar
Methylamine Salts Medium—Recipe for methylamine salts medium
Modified Chopped Meat Medium—Used for the growth of several anaerobic bacteria.
MY medium—Maltose yeast extract bacterial growth medium.
N4 Mineral Medium—Recipe for the production of N4 mineral medium.
Nitrosomonas europaea medium—Recipe for the production of Nitrosomonas europaea
medium.
Nutrient agar—Recipe for nutrient agar suitable for growth of many bacterial species.
Nutrient broth—Recipe for nutrient broth suitable for growth of many bacterial species.
MRS media—Recipe for MRS media. MRS media has been used for the recovery of lactic
acid bacteria (LAB) from various food products.
MS-Medium—Recipe for MS-medium.
N-Z amine agar with soluble starch and glucose—Agar used to grow some Actinomadura
species
NZCYM—NZ amine, NaCl, bacto-yeast extract, casamino acids, and magnesium sulfate.
NZM—NZ amine, NaCl, and magnesium sulfate.
NZYM—NZ amine, NaCl, bacto-yeast extract, and magnesium sulfate.
Oatmeal agar—agar used to grow some Actinomadura species.
Oenococcus Medium—Recipe for the preparation of Oenococcus medium.
Osmophilic Agar—Recipe for Osmophilic Agar.
Osmophilic Medium—Recipe for Osmophilic Medium.
Phenol red lactose broth—turns yellow when lactose is fermented.
Potato-Carrot Medium—agar used to grow some Actinoplanes species.
Propionibacterium Agar Recipe—Agar appropriate for the growth of Propionibacterium.
Propionibacterium Medium Recipe—Medium appropriate for the growth of
Propionibacterium.
PYS agar—agar used to grow some Actinomadura species.
R Medium—R Medium Recipe.
Rolled Oats Mineral Agar—Recipe for Rolled Oats Mineral Agar.
Saccharose agar—Recipe for the production of saccharose agar
Saccharose medium—Recipe for the production of saccharose medium
SOB media—Tryptone/yeast extract bacterial media.
SOC media—Tryptone/yeast extract bacterial media.
5% Sorbitol agar—Recipe for the production of 5% sorbitol agar.
5% Sorbitol medium—Recipe for the production of 5% sorbitol medium.
Sour Dough Medium—Recipe for the preparation of sour dough medium.
Starch-Mineral Salt (STMS) Agar—Recipe for starch-mineral salt (STMS) agar.
Styrene Mineral Salts Medium—Recipe for Styrene Mineral Salts medium.
Terrific broth—Recipe for the preparation of terrific broth bacterial media.
Thermus Agar—Recipe for agar appropriate for the growth of Therums genus
Thermus Medium—Recipe for media appropriate for the growth of Therums genus
Thiobacillus Medium F2—Recipe for the production of Thiobacillus medium F2
Tomato Juice Agar—Recipe for the preparation of tomato juice agar.
Tomato Juice Medium—Recipe for the preparation of tomato juice medium.
Tomato Juice Yeast Extract Agar—Recipe for the preparation of tomato juice yeast extract
agar.
Tomato Juice Yeast Extract Medium—Recipe for the preparation of tomato juice yeast extract
medium.
TSY agar—Trypticase soy yeast agar Recipe.
TSY broth—Trypticase soy yeast broth Recipe.
TYG Medium—Tryptone, yeast, glucose bacterial growth medium.
TYX Medium—Tryptone, yeast, xylose bacterial growth medium.
Urea Medium—Recipe for the preparation of urea medium
Uric Acid Medium—Recipe for the preparation of uric acid medium
Whey Agar—Recipe for the preparation of whey agar.
Whey Medium—Recipe for the preparation of whey medium.
Wickerham Salt Agar—Recipe for Wickerham Salt Agar.
Wickerham Salt Medium—Recipe for Wickerham Salt Medium.
Yeast Extract Glucose Medium—Yeast Extract Glucose medium recipe
YEL Agar—Recipe for YEL Agar.
YMF agar recipe—Recipe for preparation of YMF agar.
YMF medium recipe—Recipe for preparation of YMF medium.
YMG agar—Recipe for yeast and malt extract with glucose agar. This agar is used for a
number of Streptomyces species.
YMG media—Recipe for yeast and malt extract with glucose media. This media is used for a
number of Streptomyces species.
YPD Agar—Yeast extract/peptone/dextrose bacterial agar.
YPD media—Yeast extract/peptone/dextrose bacterial media.
YPG media—Yeast extract/peptone/galactose bacterial media.
YPM Agar—Recipe for YPM agar.
YPM Medium—Recipe for YPM medium.
YT (2x)—Yeast extract/tryptone bacterial media.

As mentioned, the Bacillus is cultured under conditions that allow for secretion of the antibacterial agent into the medium. In one embodiment, the conditions are such that allow for generation of a biofilm. The present inventors have uncovered particular components of a growth medium that are important for biofilm generation of bacteria being of the genus Bacillus (e.g. of the species B. subtilis). Thus, the present inventors propose that the medium used for culturing the B. subtilis comprises manganese. In another embodiment, the medium comprises glycerol. In still another embodiment, the medium comprises dextrose. In still another embodiment, the medium used for culturing comprises both manganese and dextrose.

In another embodiment, the growth medium is derived from plants. The present inventors have shown that culturing Bacilli bacteria in a plant derived medium promotes the secretion of antimicrobial agents.

In one embodiment, the plant from which the growth medium is derived is a young plant—for example a seedling or a sprout. Preferably, the plant has been grown from seed for no more than 3 days, 4 days, 5 days, 6 days 7 days or 10 days.

Exemplary plants which can be used to generate a growth medium include alfalfa sprouts (Medicago sativa) and Vigna radiate.

The plant extract may be prepared in any way known in the art. In one embodiment, the extract is a crude extract. The extract may undergo a process of sterilization and/or filtration.

The extract may be added to additional growth media in sufficient amounts such that the secretion of antimicrobial agents is enhanced.

Exemplary solid surfaces on which the culturing can be carried out include a wide range of substrates, ranging from various polymeric materials (silicone, polystyrene, polyurethane, and epoxy resins) to metals and metal oxides (silicon, titanium, aluminum, silica, and gold). Fabrication techniques (soft lithography and double casting molding techniques, microcontact printing, electron beam lithography, nanoimprint lithography, photolithography, electrodeposition methods, etc.) can be carried out on such materials in order to alter the topography of the solid surface.

Other conditions of the culture that may be altered to enhance generation of a biofilm include, but are not limited to environmental parameters such as pH, nutrient concentration, co-culture of additional bacteria and temperature.

In one embodiment, the culturing is carried out in a bioreactor.

As used herein, the term “bioreactor” refers to an apparatus adapted to support the biofilm of the invention.

The bioreactor will generally comprise one or more supports for the biofilm which may form a film thereover, and wherein the support is adapted to provide a significant surface area to enhance the formation of the biofilm. The bioreactors of the invention may be adapted for continuous throughput.

It will be appreciated that when the biofilm is generated in a bioreactor system, the conditions of the culture can be altered by altering the microfluidics (e.g. sheer stress) of the system.

As mentioned, agents or conditions are selected that bring about an advantageous change in a property of the biofilm such that the amount of the antimicrobial agent secreted into the medium is enhanced. In one embodiment, the property is the amount of biofilm. In one embodiment, the property is the thickness of biofilm. In another embodiment, the property is the density of the biofilm. In yet another embodiment, the property is the rate in which the biofilm is formed. In still another embodiment, the property is the amount of additional bacteria which is incorporated into the biofilm. In still another embodiment, the property is the resistance to temperature and/or pH.

In one embodiment, the pH of the growth substrate is higher than 6.

The culturing of this aspect of the present invention may be carried out in the presence of additional agents that serve to increase propagation of the B. subtilis bacteria and/or enhance biofilm formation. Such agents include for example acetoin.

The amount of acetoin and the timing of addition may be altered so as to promote optimal biofilm production. In one embodiment, about 0.01-5% acetoin is used. In another embodiment, about 0.01-4% acetoin is used. In another embodiment, about 0.01-3% acetoin is used. In another embodiment, about 0.01-2% acetoin is used. In another embodiment, about 0.01-1% acetoin is used. In another embodiment, about 0.01-0.5% acetoin is used.

Thus, the present inventors contemplate a culture comprising acetoin, a biofilm comprising Bacillus cells and a culture medium. In one embodiment, the culture medium is LB.

In one embodiment, about 0.05-5% acetoin is used. In another embodiment, about 0.05 4% acetoin is used. In another embodiment, about 0.05-3% acetoin is used. In another embodiment, about 0.05-2% acetoin is used. In another embodiment, about 0.05-1% acetoin is used. In another embodiment, about 0.05-0.5% acetoin is used.

In one embodiment, about 0.1-5% acetoin is used. In another embodiment, about 0.1-4% acetoin is used. In another embodiment, about 0.1-3% acetoin is used. In another embodiment, about 0.1-2% acetoin is used. In another embodiment, about 0.1-1% acetoin is used. In another embodiment, about 0.1-0.5% acetoin is used.

The cultures of this aspect of the present invention are propagated for a length of time sufficient to generate a biofilm which incorporates the B. subtilis.

In one embodiment, the conditions are selected such that the amount of biofilm produced by the B. subtilis or by the B. velezensis is enhanced and the amount of pigment secreted from the biofilm is enhanced. Alternatively, the conditions are selected such that the amount of pigment secreted by the B. subtilis is enhanced. Still alternatively, the conditions are selected such that the amount of antimicrobial secreted by the Bacillus velezensis is enhanced.

According to a particular embodiment, the color of the antimicrobial agent is a dark brown/reddish pigment.

Preferably, the B. subtilis or B. velezensis bacteria are cultured for at least 6 hours, 12 hours, 24 hours, 48 hours, 3 days, 4 days, 5 days, 6 days, 7 days, 8 days, 2 weeks, 3 weeks or longer to allow for sufficient quantities of the antimicrobial agent to be secreted.

In one embodiment, the B. subtilis or B. velezensis bacteria are cultured at a temperature between 20-40° C., more preferably between 23-32° C.—for example at about 30° C.

According to a particular embodiment, the B. subtilis bacteria are cultured in LBGM at about 30° C.

In one embodiment, the composition which is enriched in antimicrobial agents is a conditioned medium of a B. subtilis or B. velezensis culture.

Conditioned medium is the culture medium of a culture of B. subtilis or B. velezensis following a certain culturing period. The conditioned medium includes antimicrobial agents secreted by bacteria in the culture.

The culture medium can be any medium suitable for culturing the bacterial cells, as described herein above.

Following accumulation of adequate antimicrobial in the medium, the growth medium (i.e., conditioned medium) is separated from the bacterial cells and collected. It will be appreciated that the bacterial cells can be used repeatedly to condition further batches of medium over additional culture periods, provided that the cells retain their ability to condition the medium i.e. secrete antimicrobial agents.

Preferably, the conditioned medium is sterilized (e.g., filtration using a 0.2 μM filter) prior to use. The conditioned medium of some embodiments of the invention may be applied directly as an antimicrobial agent or extracted to concentrate the antimicrobial agents as further described herein above. For future use, conditioned medium is preferably stored frozen at 4° C., −20° C. or −80° C.

Additional steps may be taken to further purify the antimicrobial agents from the culture.

In one embodiment, a single antimicrobial agent is isolated from the culture.

In other embodiments, a plurality (for example 2-20 or 2-10) different antimicrobial agents are isolated from the culture.

In yet another embodiment, at least two, three, four, five or more antimicrobial agents are isolated from a single culture.

In yet another embodiment, an antimicrobial agent which comprises a color (i.e. a pigment) is isolated.

Preferably, the method for preparing the purified preparation which comprises the antimicrobial agent involves a step of removing the culture medium from the B. subtilis cells. This extracellular fraction of the liquid fermentation medium is also termed the supernatant and this fraction can be separated from the B. subtilis cellular fraction by e.g. centrifugation or filtration, or indeed by any other means available for obtaining a liquid fraction essentially without any bacterial cells present therein.

In particular embodiments of the invention, the purification comprises at least one size fractionation step. Preferably, this size fractionation step is performed on the extracellular fraction.

This size fractionation step may ensure that every component of the composition has a molecular weight of at least a given value. The size fractionation step may be any size fraction known to the skilled person, for example ultracentrifugation, ultrafiltration, microfiltration or gel-filtration.

Thus in a particular embodiment of the invention, the agent is purified from a liquid growth medium by a method involving one or more purification steps selected from the group consisting of ultracentrifugation, ultrafiltration, microfiltration and gel-filtration. Preferably, the purification step(s) are selected from the group consisting of ultrafiltration, microfiltration and ultracentrifugation, even more preferably from the group consisting of ultrafiltration and microfiltration.

Ultrafiltration is a membrane process where the membrane fractionates components of a liquid according to size. The membrane configuration is normally cross-flow wherein the liquid containing the relevant components are flowing across the membrane. Some of the liquid, containing components smaller than the nominal pore size of the membrane will permeate through the membrane. Molecules larger than the nominal pore size will be retained. The desired product may be in the retentate or the filtrate. If the ultrafiltration is performed in order to prepare a composition, wherein every agent within the composition has a molecular weight above a given value, the desired product is in the retentate. If a serial fractionation is made, the product may be in the retentate or filtrate.

Microfiltration is a membrane separation process similar to UF but with even larger membrane pore size allowing larger particles to pass through.

Gel filtration is a chromatographic technique in which particles are separated according to size. The filtration medium will typically be small gel beads which will take up the molecules that can pass through the bead pores. Larger molecules will pass through the column without being taken up by the beads.

Gel-filtration, ultrafiltration or microfiltration may for example be performed as described in R Hatti-Kaul and B Mattiasson (2001), Downstream Processing in Biotechnology, in Basic Biotechnology, eds C Ratledge and B Kristiansen, Cambridge University Press) pp 189.

In another embodiment the antimicrobial agent in the medium may be isolated by precipitation, such as precipitation with alcohol, such as ethanol and/or chromatographic methods. This may for example be performed essentially as described in WO2003/020944. It is also contemplated within the invention that the antimicrobial agents are isolated by sequentially performing two or more of above-mentioned methods. By way of example the antimicrobial agent may be isolated by first performing a size fractionation step followed by precipitation.

Other methods for isolating the antimicrobial agent of this aspect of the present invention are also contemplated by the present inventors including but not limited to HPLC.

Preferably more than 10% of the all the components of the purified preparation derived from the B. subtilis or B. velezensis bacteria comprises antimicrobial activity. Preferably, more than 20% of all the components of the purified preparation derived from the B. subtilis or B. velezensis bacteria comprises antimicrobial activity. In one embodiment, more than 30% of all the components of the purified preparation derived from the B. subtilis or B. velezensis bacteria comprises antimicrobial activity. In one embodiment, more than 40% of all the components of the purified preparation derived from the B. subtilis or B. velezensis bacteria comprises antimicrobial activity. In one embodiment, more than 50% of all the components of the purified preparation derived from the B. subtilis bacteria comprises antimicrobial activity.

Preferably more than 10% of the B. subtilis-derived components or B. velezensis-derived components of the purified preparation comprise antimicrobial activity. Preferably more than 20% of the B. subtilis-derived components or B. velezensis-derived components of the purified preparation comprise antimicrobial activity. In one embodiment, more than 30% of the B. subtilis-derived components or B. velezensis-derived components of the purified preparation comprise antimicrobial activity. In one embodiment, more than 40% of the B. subtilis-derived components or B. velezensis-derived components of the purified preparation comprise antimicrobial activity. In one embodiment, more than 50% of the B. subtilis-derived components or B. velezensis-derived components of the purified preparation comprise antimicrobial activity. Following isolation of the antimicrobial agent, the activity thereof may be tested.

In one embodiment, the at least one antimicrobial agent is between 1-100% of the B. subtilis or B. velezensis components in the composition of matter (per weight), between 5-100% of the B. subtilis or B. velezensis components in the composition of matter (per weight), between 10-100% of the B. subtilis or B. velezensis components in the composition of matter (per weight), between 15-100% of the B. subtilis or B. velezensis components in the composition of matter (per weight), between 20-100% of the B. subtilis or B. velezensis components in the composition of matter (per weight), between 25-100% of the B. subtilis or B. velezensis components in the composition of matter (per weight), between 30-100% of the B. subtilis or B. velezensis components in the composition of matter (per weight), between 35-100% of the B. subtilis or B. velezensis components in the composition of matter (per weight), between 40-100% of the B. subtilis or B. velezensis components in the composition of matter (per weight), between 45-100% of the B. subtilis or B. velezensis components in the composition of matter, between 50-100% of the B. subtilis or B. velezensis components in the composition of matter (per weight), between 55-100% of the B. subtilis or B. velezensis components in the composition of matter (per weight), between 60-100% of the B. subtilis or B. velezensis components in the composition of matter (per weight), between 65-100% of the B. subtilis or B. velezensis components in the composition of matter (per weight), or even between 70-100% of the B. subtilis or B. velezensis components in the composition of matter.

In vitro assays that can be used for confirming the antimicrobial activity of the purified fractions include, for example, the addition of varying concentrations of the antimicrobial composition to paper disks and placing the disks on agar containing a suspension of the pathogen of interest. Following incubation, clear inhibition zones develop around the discs that contain an effective concentration of the antimicrobial polypeptide (Liu et al. (1994) Plant Biology 91:1888-1892, herein incorporated by reference). Additionally, microspectrophotometrical analysis can be used to measure the in vitro antimicrobial properties of a composition (Hu et al. (1997) Plant Mol. Biol. 34:949-959 and Cammue et al. (1992) J. Biol. Chem. 267: 2228-2233, both of which are herein incorporated by reference). Assays that specifically measure antibacterial activity are also well known in the art. See, for example, Clinical and Laboratory Standards Institute, Guideline M7-A6, Methods for dilution antimicrobial susceptibility tests for bacteria that grow aerobically, herein incorporated by reference.

Since the agents of the present invention comprise anti-microbial properties they may be used to kill microbes.

Thus, according to another aspect of the present invention there is provided a method of killing a microbe, the method comprising contacting the microbe with the antimicrobial agents of the present invention.

As used herein the term “contacting” refers to the positioning of the agents of the present invention such that they are in direct or indirect contact with the bacterial cells. Thus, the present invention contemplates both applying the agents of the present invention to a desirable surface and/or directly to the bacterial cells.

Contacting surfaces with the agents described herein can be effected using any method known in the art including spraying, spreading, wetting, immersing, dipping, painting, ultrasonic welding, welding, bonding or adhering. The agents of the present invention may be attached to a solid surface as monolayers or multiple layers.

The present invention envisages coating a wide variety of surfaces with the agents of the present invention including fabrics, fibers, foams, films, concretes, masonries, glass, metals, plastics, polymers, and like.

An exemplary solid surface that may be coated with the agents of the present invention is an intracorporal or extra-corporeal medical device or implant.

An “implant” as used herein refers to any object intended for placement in a human body that is not a living tissue. The implant may be temporary or permanent. Implants include naturally derived objects that have been processed so that their living tissues have been devitalized. As an example, bone grafts can be processed so that their living cells are removed (acellularized), but so that their shape is retained to serve as a template for ingrowth of bone from a host. As another example, naturally occurring coral can be processed to yield hydroxyapatite preparations that can be applied to the body for certain orthopedic and dental therapies. An implant can also be an article comprising artificial components.

Thus, for example, the present invention therefore envisions coating vascular stents with the agents of the present invention. Another possible application of the agents of the present invention is the coating of surfaces found in the medical and dental environment.

Surfaces found in medical environments include the inner and outer aspects of various instruments and devices, whether disposable or intended for repeated uses. Examples include the entire spectrum of articles adapted for medical use, including scalpels, needles, scissors and other devices used in invasive surgical, therapeutic or diagnostic procedures; blood filters, implantable medical devices, including artificial blood vessels, catheters and other devices for the removal or delivery of fluids to patients, artificial hearts, artificial kidneys, orthopedic pins, plates and implants; catheters and other tubes (including urological and biliary tubes, endotracheal tubes, peripherally insertable central venous catheters, dialysis catheters, long term tunneled central venous catheters peripheral venous catheters, short term central venous catheters, arterial catheters, pulmonary catheters, Swan-Ganz catheters, urinary catheters, peritoneal catheters), urinary devices (including long term urinary devices, tissue bonding urinary devices, artificial urinary sphincters, urinary dilators), shunts (including ventricular or arterio-venous shunts); prostheses (including breast implants, penile prostheses, vascular grafting prostheses, aneurysm repair devices, heart valves, artificial joints, artificial larynxes, otological implants), anastomotic devices, vascular catheter ports, clamps, embolic devices, wound drain tubes, hydrocephalus shunts, pacemakers and implantable defibrillators, and the like. Other examples will be readily apparent to practitioners in these arts.

Surfaces found in the medical environment include also the inner and outer aspects of pieces of medical equipment, medical gear worn or carried by personnel in the health care setting. Such surfaces can include counter tops and fixtures in areas used for medical procedures or for preparing medical apparatus, tubes and canisters used in respiratory treatments, including the administration of oxygen, of solubilized drugs in nebulizers and of anesthetic agents. Also included are those surfaces intended as biological barriers to infectious organisms in medical settings, such as gloves, aprons and faceshields. Commonly used materials for biological barriers may be latex-based or non-latex based. Vinyl is commonly used as a material for non-latex surgical gloves. Other such surfaces can include handles and cables for medical or dental equipment not intended to be sterile.

Additionally, such surfaces can include those non-sterile external surfaces of tubes and other apparatus found in areas where blood or body fluids or other hazardous biomaterials are commonly encountered.

Other surfaces related to health include the inner and outer aspects of those articles involved in water purification, water storage and water delivery, and those articles involved in food processing. Thus the present invention envisions coating a solid surface of a food or beverage container to extend the shelf life of its contents.

Surfaces related to health can also include the inner and outer aspects of those household articles involved in providing for nutrition, sanitation or disease prevention. Examples can include food processing equipment for home use, materials for infant care, tampons and toilet bowls.

In addition, the agents of the present invention may have veterinary applications including disinfection of animal cages, coops or homes.

It will be appreciated that since the agents of the present invention have antimicrobial activity, the present invention contemplates use thereof for treating infection in a mammalian subject (e.g. humans).

Typically, the subjects who are treated are infected with pathogens which cause a disease.

As used herein, the term “pathogen” refers to a microbe or microorganism such as a virus, bacterium, prion or fungus that causes a disease (e.g. a respiratory disease).

According to a particular embodiment, the pathogen is a human pathogen.

Exemplary pathogenic viruses may belong to the following families: Adenoviridae, Coronaviridae, Picornaviridae, Herpesviridae, Hepadnaviridae, Flaviviridae, Retroviridae, Orthomyxoviridae, Paramyxoviridae, Papovaviridae, Polyomavirus, Rhabdoviridae, Togaviridae. Particular pathogenic viruses contemplated by the present invention are those that cause smallpox, influenza, mumps, measles, chickenpox, ebola, or rubella.

According to a particular embodiment, the virus is one which brings about a respiratory infection (e.g. an upper respiratory tract infection and/or a lower respiratory tract infection).

Thus, according to a particular embodiment, the pathogenic virus is an influenza virus (e.g. influenza virus A—(e.g. HIN1, H2N2, H3N2, H5N1, H7N7, H1N2, H9N2, H7N2, H7N3, H10N7 and H7N9), influenza virus B or influenza virus C).

In another embodiment, the pathogenic virus is a parainfluenza virus (hPIV) including the human parainfluenza virus type 1 (hPIV-1) (causes croup); the human parainfluenza virus type 2 (hPIV-2) (causes croup and other upper and lower respiratory tract illnesses), the human parainfluenza virus type 3 (hPIV-3) (associated with bronchiolitis and pneumonia) and the human parainfluenza virus type 4 (hPIV-4).

In yet another embodiment, the pathogenic virus is a respiratory syncytial virus (RSV).

In still another embodiment, the pathogenic virus is a coronavirus.

Exemplary pathogenic bacteria include Mycobacterium tuberculosis which causes tuberculosis, Streptococcus and Pseudomonas which cause pneumonia, and Shigella, Campylobacter and Salmonella which cause foodborne illnesses. Other exemplary pathogenic bacteria contemplated by the present invention are those that cause infections such as tetanus, typhoid fever, diphtheria, syphilis and Hansen's disease.

According to a particular embodiment, the pathogenic bacteria is E. coli, Klebsiella pneumonia, Enterococcus faecalis, Staphylococcus aureus (MSSA, MRSA), Salmonella Enteritidis or Serratia marcescens.

According to one embodiment, the infection is an acute infection.

According to another embodiment, the infection is a chronic infection.

According to one embodiment, the agents are used to treat a topical infection (i.e. infection of the skin) and are provided in a topical formulation.

According to another embodiment, the agents are used to treat an infection inside the body.

In this case, the agents may be provided ex vivo or in vivo.

Accordingly, the present invention contemplates contacting cells with the agents per se or as part of a pharmaceutical composition.

In one embodiment, the pharmaceutical compositions of the present invention are administered to a subject in need thereof in order to prevent or treat a bacterial infection.

As used herein, the term “subject in need thereof” refers to a mammal, preferably a human subject.

As used herein, the term “treating” refers to curing, reversing, attenuating, alleviating, minimizing, suppressing or halting the deleterious effects of a pathogen infection.

The phrase “pharmaceutical composition”, as used herein refers to a preparation of one or more of the active ingredients described herein with other chemical components such as physiologically suitable carriers and excipients. The purpose of a pharmaceutical composition is to facilitate administration of a compound to an organism.

As used herein the term “active ingredient” refers to the agents of the present invention accountable for the intended biological effect.

Hereinafter, the phrases “physiologically acceptable carrier” and “pharmaceutically acceptable carrier”, which may be used interchangeably, refer to a carrier or a diluent that does not cause significant irritation to an organism and does not abrogate the biological activity and properties of the administered compound. An adjuvant is included under these phrases.

Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils, and polyethylene glycols.

Techniques for formulation and administration of drugs may be found in the latest edition of “Remington's Pharmaceutical Sciences”, Mack Publishing Co., Easton, PA, which is herein fully incorporated by reference and are further described herein below.

It will be appreciated that the agents of the present invention can be provided to the individual with additional active agents to achieve an improved therapeutic effect as compared to treatment with each agent by itself.

Exemplary additional agents include antibiotics (e.g. rifampicin, chloramphenicol and spectinomycin), antibacterial peptides, antivirals, antifungals etc.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the invention may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount.

Capsules and cartridges of, e.g., gelatin for use in a dispenser may be formulated containing a powder mix of the compound and a suitable powder base such as lactose or starch.

The preparations described herein may be formulated for parenteral administration, e.g., by bolus injection or continuous infusion. Formulations for injection may be presented in unit dosage form, e.g., in ampoules or in multidose containers with optionally, an added preservative.

The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The preparation of the present invention may also be formulated in rectal compositions such as suppositories or retention enemas, using, e.g., conventional suppository bases such as cocoa butter or other glycerides.

The preparation of the present invention may also be formulated as a topical compositions, such as a spray, a cream, a mouthwash, a wipe, a foam, a soap, an oil, a solution, a lotion, an ointment, a paste and a gel.

Pharmaceutical compositions suitable for use in context of the present invention include compositions wherein the active ingredients are contained in an amount effective to achieve the intended purpose. More specifically, a therapeutically effective amount means an amount of active ingredients effective to prevent, alleviate or ameliorate symptoms of disease or prolong the survival of the subject being treated.

Determination of a therapeutically effective amount is well within the capability of those skilled in the art.

For any preparation used in the methods of the invention, the therapeutically effective amount or dose can be estimated initially from in vitro assays. For example, a dose can be formulated in animal models and such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. [See e.g., Fingl, et al., (1975) “The Pharmacological Basis of Therapeutics”, Ch. 1 p.1].

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

The present invention further contemplate killing microbes which are present on plants with the compositions which comprise the isolated antibacterial agents of the present invention, or alternatively the Bacillus bacteria described herein (for example that having a 16S rRNA sequence as set forth in SEQ ID NO: 1, 2 or 3).

Thus, the isolated agents or microbes themselves may be used for use in controlling plant diseases (i.e. at the pre-harvest stage).

As used herein the term “plant” refers to whole plants, a plant tissue, a plant organ, a fruit, a vegetable, an eatable portion of a plant, a grafted plant including seeds, shoots, stems, roots (including tubers), rootstock, scion, and plant cells, tissues, fruit, flower and organs. The plant may be in any form including cuttings and harvested material (e.g., fruit).

In one embodiment, the plant is an agricultural plant.

The phrase “agricultural plants”, or “plants of agronomic importance”, refers to plants that are cultivated by humans for food, feed, fiber, and fuel purposes. In one embodiment, the plant is not a wild plant.

In one embodiment, a monocotyledonous plant is treated. Monocotyledonous plants belong to the orders of the Alismatales, Arales, Arecales, Bromeliales, Commelinales, Cyclanthales, Cyperales, Eriocaulales, Hydrocharitales, Juncales, Lilliales, Najadales, Orchidales, Pandanales, Poales, Restionales, Triuridales, Typhales, and Zingiberales. Plants belonging to the class of the Gymnospermae are Cycadales, Ginkgoales, Gnetales, and Pinales. In a particular embodiment, the monocotyledonous plant can be selected from the group consisting of a maize, rice, wheat, barley, and sugarcane.

In another embodiment, a dicotyledonous plant is treated, including those belonging to the orders of the Aristochiales, Asterales, Batales, Campanulales, Capparales, Caryophyllales, Casuarinales, Celastrales, Cornales, Diapensales, Dilleniales, Dipsacales, Ebenales, Ericales, Eucomiales, Euphorbiales, Fabales, Fagales, Gentianales, Geraniales, Haloragales, Hamamelidales, Middles, Juglandales, Lamiales, Laurales, Lecythidales, Leitneriales, Magniolales, Malvales, Myricales, Myrtales, Nymphaeales, Papeverales, Piperales, Plantaginales, Plumb aginales, Podostemales, Polemoniales, Polygalales, Polygonales, Primulales, Proteales, Rafflesiales, Ranunculales, Rhamnales, Rosales, Rubiales, Salicales, Santales, Sapindales, Sarraceniaceae, Scrophulariales, Theales, Trochodendrales, Umbellales, Urticales, and Violates. In a particular embodiment, the dicotyledonous plant can be selected from the group consisting of cotton, bean, pepper, and tomato.

Preferably, the plant is an agricultural plant. Agricultural plants include monocotyledonous species such as: maize (Zea mays), common wheat (Triticum aestivum), spelt (Triticum spelta), einkorn wheat (Triticum monococcum), emmer wheat (Triticum dicoccum), durum wheat (Triticum durum), Asian rice (Oryza sativa), African rice (Oryza glabaerreima), wild rice (Zizania aquatica, Zizania latifolia, Zizania palustris, Zizania texana), barley (Hordeum vulgare), Sorghum (Sorghum bicolor), Finger millet (Eleusine coracana), Proso millet (Panicum miliaceum), Pearl millet (Pennisetum glaucum), Foxtail millet (Setaria italica), Oat (Avena sativa), Triticale (Triticosecale), rye (Secale cereal), Russian wild rye (Psathyrostachys juncea), bamboo (Bambuseae), or sugarcane (e.g., Saccharum arundinaceum, Saccharum barberi, Saccharum bengalense, Saccharum edule, Saccharum munja, Saccharum officinarum, Saccharum procerum, Saccharum ravennae, Saccharum robustum, Saccharum sinense, or Saccharum spontaneum); as well as dicotyledonous species such as: soybean (Glycine max), canola and rapeseed cultivars (Brassica napus), cotton (genus Gossypium), alfalfa (Medicago sativa), cassava (genus Manihot), potato (Solanum tuberosum), tomato (Solanum lycopersicum), pea (Pisum sativum), chick pea (Cicer arietinum), lentil (Lens culinaris), flax (Linum usitatissimum) and many varieties of vegetables.

The isolated antimicrobial agents (or the bacteria themselves) can be applied to plants by spraying, dusting, coating, soaking, irrigation, drenching or otherwise treating them with the active ingredients or alternatively, by treating with the active ingredients the plant seeds, the soil around the plant, or the soil, rice pads or the water for hydroponic culture where the seeds are to be sown.

The application may be affected either before or after the plant is infected with a pathogen.

According to an embodiment, the regimen is performed such as to control the spread of a pathogen and/or eliminate a pathogen and/or eliminate/reduce/minimize any damage that can be caused by the pathogen.

The antimicrobial agents (or the bacteria themselves) can be formulated as dips, sprays, seed coatings or concentrates.

Thus, according to another aspect of the present invention there is provided a method for prolonging the shelf life of a plant, the post-harvest quality of a plant, increasing a plant performance parameter, or a combination thereof comprising pre-harvest contacting said plant with an effective amount of Bacillus bacteria having a 16S rRNA sequence as set forth in SEQ ID NOs: 1, 2 or 3, thereby prolonging the shelf life of a plant, the post-harvest quality of a plant, or a combination thereof.

The shelf-life may be improved by at least 1 day, 2 days, 3 days, four days, five days, six days, 7 days, 10 days or more.

It will be appreciated that by controlling (i.e. reducing) pathogenic infection in the plant, the post-harvest quality of the plant is improved and losses are reduced. In one embodiment, the amount of bacteria which can cause disease in humans is reduced, thereby improving the safety of the plant for consumption. Concurrently, or alternatively, plant performance parameters may also be improved (including, but not limited to growth parameters, crop production, and pathogenic resistance).

The contacting may be effected at any stage of the plant life cycle e.g. a post-blossom stage, a blossom stage, a pre-blossom stage, or any combination thereof.

The contacting may be effected in the vicinity of or onto: a root, a stem, a trunk, a seed, a fruit, a flower, a leaf, or any combination thereof of the plant.

According to an embodiment, application is carried out in an open field. According to an embodiment, application is carried out in a greenhouse. According to an embodiment, the application is carried out once. According to an embodiment, at least two applications are carried out at any regimen or duration.

According to an embodiment, the applying comprises repeated application (2 or more applications e.g., every week, seasonal, bi-weekly, bi-monthly etc.). Repeated applications are especially envisaged for field/greenhouse treatments.

According to an embodiment, repeated application comprises weekly, daily, monthly, or bi-monthly administration during blossom, post-blossom, pre-blossom, or any combination thereof. For example, suggested regimen may include but is not limited to, spraying plants in open fields and green house, adding to irrigation of plants grown in the open field, green house and in pots.

As well as pre-harvest treatment, the present inventors contemplate post-harvest treatment with the Bacillus bacteria described herein or antimicrobial agents derived therefrom.

Thus, according to still another aspect of the present invention there is provided a method for prolonging the shelf life of a plant or the post-harvest quality of a plant, or a combination thereof comprising post-harvest contacting said plant with an effective amount of Bacillus bacteria of the species subtilis or velezensis, thereby prolonging the shelf life of a plant or the post-harvest quality of a plant, or a combination thereof.

The post-harvest contacting may be carried out no more than 6 hours following harvesting, no more than 12 hours following harvesting, no more than 1 day following harvesting, no more than 2 days following harvesting, no more than 3 days following harvesting, or no more than 4 day following harvesting,

According to an embodiment, the post-harvest contacting is carried out in a storage facility (e.g., dark room, refrigerator).

The contacting may comprise dipping the whole foliage branch in the solution post-harvest, adding to vase of cut flowers before and/or after harvest and possibly before shipment.

Since the antimicrobial agents derived from the Bacillus bacteria were shown to be effective at killing bacteria which are pathogenic to humans, the present inventors contemplate that the Bacillus bacteria (and agents isolated therefrom) may be used as a preservative in the food industry in general (and not just for plant-based materials).

Thus, according to still another aspect of the present invention, there is provided a method of preserving a food product comprising contacting the food with a Bacillus bacteria of the species Subtilis or Velezensis or an agent derived from said Bacillus bacteria under conditions that down-regulate an activity and/or an amount of at least one type of microbe that is capable of spoiling the food product, thereby preserving the food product.

In one embodiment, the food is a plant derived food. In another embodiment, the food is an animal derived food. In still another embodiment, the food is a dairy food. In still another embodiment, the food is a poultry food.

The Bacillus bacteria described herein (or anti-microbial agents isolated therefrom) may be provided per se, or may be formulated with an agriculturally acceptable carrier.

As used herein the term “agriculturally acceptable carrier” refers to a material that facilitates application of the bacteria (or agent isolated therefrom) to the intended target, which may be for example a plant, a plant material, compost, earth, surroundings or equipment, or that facilitates storage, transport or handling. Carriers used in compositions for application to plants and plant material are preferably non-phytotoxic or only mildly phytotoxic. A suitable carrier may be a solid, liquid or gas depending on the desired formulation. In one embodiment the carriers include polar liquid carriers such as water, mineral oils and vegetable oils. In one embodiment the carrier enhances the stability of the active ingredient as described herein.

The carrier can include a dispersant, a surfactant, an additive, water, a thickener, an anti-caking agent, residue breakdown, a composting formulation, a granular application, diatomaceous earth, an oil, a coloring agent, a stabilizer, a preservative, a polymer, a coating, or a combination thereof. One of ordinary skill in the art can readily determine the appropriate carrier to be used taking into consideration factors such as a particular bacterial strain, plant to which the bacteria is to be applied, type of soil, climate conditions, whether the bacteria is in liquid, solid or powder form, and the like.

The additive can comprise an oil, a gum, a resin, a clay, a polyoxyethylene glycol, a terpene, a viscid organic, a fatty acid ester, a sulfated alcohol, an alkyl sulfonate, a petroleum sulfonate, an alcohol sulfate, a sodium alkyl butane diamate, a polyester of sodium thiobutant dioate, a benzene acetonitrile derivative, a proteinaceous material, or a combination thereof.

The surfactant can contain a heavy petroleum oil, a heavy petroleum distillate, a polyol fatty acid ester, a polyethoxylated fatty acid ester, an aryl alkyl polyoxyethylene glycol, an alkyl amine acetate, an alkyl aryl sulfonate, a polyhydric alcohol, an alkyl phosphate, or a combination thereof.

The anti-caking agent can include a sodium salt such as a sodium sulfite, a sodium sulfate, a sodium salt of monomethyl naphthalene sulfonate, a sodium salt of dimethyl naphthalene sulfonate, or a combination thereof; or a calcium salt such as calcium carbonate, diatomaceous earth, or a combination thereof.

Exemplary agriculturally acceptable carriers include, but are not limited to, vermiculite, charcoal, sugar factory carbonation press mud, rice husk, carboxymethyl cellulose, peat, perlite, fine sand, calcium carbonate, flour, alum, a starch, talc, polyvinyl pyrrolidone, or a combination thereof.

The Bacillus cultures can be prepared as solid, liquid. emulsion or powdered formulations as is known in the art. The cultures of the present invention can be formulated as a seed coating formulation, a liquid formulation for application to plants or to a plant growth medium, or a solid formulation for application to plants or to a plant growth medium.

When the Bacillus culture is prepared as a liquid formulation for application to plants or to a plant growth medium, it can be prepared in a concentrated formulation or a working form formulation. In some instances, the seed coating formulation of the present invention is an aqueous or oil-based solution for application to seeds.

When the Bacillus culture of the present invention is prepared as a solid formulation for application to plants or to a plant growth medium, it can be prepared as a granular formulation or a powder agent. The seed coating formulation can be a powder or granular formulation for application to seeds.

The Bacillus culture (or antimicrobial agent isolated therefrom) can further include an agrochemical (i.e. an agent that promotes the growth of a plant).

Thus, according to another aspect of the present invention there is provided an article of manufacture comprising Bacillus bacteria which comprise a 16S rRNA nucleic acid sequence as set forth in SEQ ID NO: 1, 2 or 3 and an agent which promotes the growth of a plant.

Examples of agents that promote the growth of a plant include a fertilizer, a micronutrient fertilizer material, an insecticide, a herbicide, a plant growth regulator, an acaricide, a rodenticide, a fungicide, a nutrient, a molluscicide, an algicide, a pesticide, a fungal inoculant, or a combination thereof.

In some instances, the fertilizer is a liquid fertilizer. The agrochemical can either be applied to a plant growth medium or to plants and/or seeds. Liquid fertilizer can include, without limitation, ammonium sulfate, ammonium nitrate, ammonium sulfate nitrate, ammonium chloride, ammonium bisulfate, ammonium polysulfide, ammonium thiosulfate, aqueous ammonia, anhydrous ammonia, ammonium polyphosphate, aluminum sulfate, calcium nitrate, calcium ammonium nitrate, calcium sulfate, calcined magnesite, calcitic limestone, calcium oxide, calcium nitrate, dolomitic limestone, hydrated lime, calcium carbonate, diammonium phosphate, monoammonium phosphate, magnesium nitrate, magnesium sulfate, potassium nitrate, potassium chloride, potassium magnesium sulfate, potassium sulfate, sodium nitrates, magnesian limestone, magnesia, urea, urea-formaldehydes, urea ammonium nitrate, sulfur-coated urea, polymer-coated urea, isobutylidene diurea, K₂SO₄—2MgSO₄, kainite, sylvinite, kieserite, Epsom salts, elemental sulfur, marl, ground oyster shells, fish meal, oil cakes, fish manure, blood meal, rock phosphate, super phosphates, slag, bone meal, wood ash, manure, bat guano, peat moss, compost, green sand, cottonseed meal, feather meal, crab meal, fish emulsion, or a combination thereof.

The micronutrient fertilizer material can comprise boric acid, a borate, a boron frit, copper sulfate, a copper frit, a copper chelate, a sodium tetraborate decahydrate, an iron sulfate, an iron oxide, iron ammonium sulfate, an iron frit, an iron chelate, a manganese sulfate, a manganese oxide, a manganese chelate, a manganese chloride, a manganese frit, a sodium molybdate, molybdic acid, a zinc sulfate, a zinc oxide, a zinc carbonate, a zinc frit, zinc phosphate, a zinc chelate, or a combination thereof.

The insecticide can include an organophosphate, a carbamate, a pyrethroid, an acaricide, an alkyl phthalate, boric acid, a borate, a fluoride, sulfur, a haloaromatic substituted urea, a hydrocarbon ester, a biologically-based insecticide, or a combination thereof.

The herbicide can comprise a chlorophenoxy compound, a nitrophenolic compound, a nitrocresolic compound, a dipyridyl compound, an acetamide, an aliphatic acid, an anilide, a benzamide, a benzoic acid, a benzoic acid derivative, anisic acid, an anisic acid derivative, a benzonitrile, benzothiadiazinone dioxide, a thiocarbamate, a carbamate, a carbanilate, chloropyridinyl, a cyclohexenone derivative, a dinitroaminobenzene derivative, a fluorodinitrotoluidine compound, isoxazolidinone, nicotinic acid, isopropylamine, an isopropylamine derivative, oxadiazolinone, a phosphate, a phthalate, a picolinic acid compound, a triazine, a triazole, a uracil, a urea derivative, endothall, sodium chlorate, or a combination thereof.

The fungicide can comprise a substituted benzene, a thiocarbamate, an ethylene bis dithiocarbamate, a thiophthalidamide, a copper compound, an organomercury compound, an organotin compound, a cadmium compound, anilazine, benomyl, cyclohexamide, dodine, etridiazole, iprodione, metlaxyl, thiamimefon, triforine, or a combination thereof.

The fungal inoculant can comprise a fungal inoculant of the family Glomeraceae, a fungal inoculant of the family Claroidoglomeraceae, a fungal inoculant of the family Gigasporaceae, a fungal inoculant of the family Acaulosporaceae, a fungal inoculant of the family Sacculosporaceae, a fungal inoculant of the family Entrophosporaceae, a fungal inoculant of the family Pacidsporaceae, a fungal inoculant of the family Diversisporaceae, a fungal inoculant of the family Paraglomeraceae, a fungal inoculant of the family Archaeosporaceae, a fungal inoculant of the family Geosiphonaceae, a fungal inoculant of the family Ambisporaceae, a fungal inoculant of the family Scutellosporaceae, a fungal inoculant of the family Dentiscultataceae, a fungal inoculant of the family Racocetraceae, a fungal inoculant of the phylum Basidiomycota, a fungal inoculant of the phylum Ascomycota, a fungal inoculant of the phylum Zygomycota, or a combination thereof.

In one embodiment, the plant growth regulator is selected from the group consisting of: Abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat (mepiquat chloride), naphthaleneacetic acid, N-6-benzyladenine, paclobutrazol, prohexadione (prohexadione-calcium), prohydrojasmon, thidiazuron, triapenthenol, tributyl phosphorotrithioate, 2,3,5-tri-iodobenzoic acid, trinexapac-ethyl and uniconazole. Other examples of plant growth regulators which can be comprised in the article of manufacture include those based on dichlorophene and benzylalcohol hemi formal (Proxel® from ICI or Acticide® RS from Thor Chemie and Kathon® MK from Rohm & Haas) and isothiazolinone derivatives such as alkylisothiazolinones and benzisothiazolinones (Acticide® MBS from Thor Chemie). Other plant growth regulators that can be incorporated seed coating compositions are described in US 2012/0108431, which is incorporated by reference in its entirety.

Preferred nematode-antagonistic biocontrol agents include ARF18; Arthrobotrys spp.; Chaetomium spp.; Cylindrocarpon spp.; Exophilia spp.; Fusarium spp.; Gliocladium spp.; Hirsutella spp.; Lecanicillium spp.; Monacrosporium spp.; Myrothecium spp.; Neocosmospora spp.; Paecilomyces spp.; Pochonia spp.; Stagonospora spp.; vesicular-arbuscular mycorrhizal fungi, Burkholderia spp.; Pasteuria spp., Brevibacillus spp.; Pseudomonas spp.; and Rhizobacteria.

Particularly preferred nematode-antagonistic biocontrol agents include ARF18, Arthrobotrys oligospora, Arthrobotrys dactyloides, Chaetomium globosum, Cylindrocarpon heteronema, Exophilia jeanselmei, Exophilia pisciphila, Fusarium aspergilus, Fusarium solani, Gliocladium catenulatum, Gliocladium roseum, Gliocladium vixens, Hirsutella rhossiliensis, Hirsutella minnesotensis, Lecanicillium lecanii, Monacrosporium drechsleri, Monacrosporium gephyropagum, Myrotehcium verrucaria, Neocosmospora vasinfecta, Paecilomyces lilacinus, Pochonia chlamydosporia, Stagonospora heteroderae, Stagonospora phaseoli, vesicular-arbuscular mycorrhizal fungi, Burkholderia cepacia, Pasteuria penetrans, Pasteuria thornei, Pasteuria nishizawae, Pasteuria ramosa, Pastrueia usage, Brevibacillus laterosporus strain G4, Pseudomonas fluorescens and Rhizobacteria.

In another embodiment, the article of manufacture can comprise a nutrient. The nutrient can be selected from the group consisting of a nitrogen fertilizer including, but not limited to Urea, Ammonium nitrate, Ammonium sulfate, Non-pressure nitrogen solutions, Aqua ammonia, Anhydrous ammonia, Ammonium thiosulfate, Sulfur-coated urea, Urea-formaldehydes, IBDU, Polymer-coated urea, Calcium nitrate, Ureaform, and Methylene urea, phosphorous fertilizers such as Diammonium phosphate, Monoammonium phosphate, Ammonium polyphosphate, Concentrated superphosphate and Triple superphosphate, and potassium fertilizers such as Potassium chloride, Potassium sulfate, Potassium-magnesium sulfate, Potassium nitrate. Such compositions can exist as free salts or ions within the seed coat composition. Alternatively, nutrients/fertilizers can be complexed or chelated to provide sustained release over time.

In one embodiment, the article of manufacture may comprise a rodenticide selected from the group of substances consisting of 2-isovalerylindan-1,3-dione, 4-(quinoxalin-2-ylamino) benzenesulfonamide, alpha-chlorohydrin, aluminum phosphide, antu, arsenous oxide, barium carbonate, bisthiosemi, brodifacoum, bromadiolone, bromethalin, calcium cyanide, chloralose, chlorophacinone, cholecalciferol, coumachlor, coumafuryl, coumatetralyl, crimidine, difenacoum, difethialone, diphacinone, ergocalciferol, flocoumafen, fluoroacetamide, flupropadine, flupropadine hydrochloride, hydrogen cyanide, iodomethane, lindane, magnesium phosphide, methyl bromide, norbormide, phosacetim, phosphine, phosphorus, pindone, potassium arsenite, pyrinuron, scilliroside, sodium arsenite, sodium cyanide, sodium fluoroacetate, strychnine, thallium sulfate, warfarin and zinc phosphide.

For any of the plant-related or food-related aspects described herein above, the present inventors further contemplate application of chlorine. The chlorine is preferably in a solid form—for example as calcium hypochlorite. The bacterial compositions and antimicrobial products described herein may be co-applied (in the same composition i.e. co-formulated, or in separate formulations), may be applied prior to or following application of the chlorine-based product. In one embodiment, the amount of chlorine based product added is less than the amount which is typically administered since the bacterial component may act synergistically with the chlorine-based product. In one embodiment, about 20,000 ppm of calcium hypochlorite is added, about 10,000 ppm of calcium hypochlorite is added or about 5,000 ppm of calcium hypochlorite is added.

Thus, the amount of calcium hypochlorite may be between 5,000 ppm to 50,000 ppm, 5,000 ppm to 40,000 ppm, 5,000 ppm to 30,000 ppm, 5,000 ppm to 20,000 ppm, 5,000 ppm to 10,000 ppm.

According to a particular embodiment, the food is a sprout (e.g. alfalfa sprout) or a seed.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment.

Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, C A (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

Materials and Methods

Bacterial cultures were maintained as glycerol stocks and stored at −80° C. For each experiment, a fresh culture was made and bacteria were grown in Lysogeny broth Luria-Bertani for 18-20 h at 37° C. with shaking (150 rpm) to obtain stationary phase cultures. Biofilm generation and pigment secretion of Bacillus subtilis (3610) and tested mutants were achieved at 30° C. in the biofilm-promoting medium LBGM (LB plus 1% [vol/vol] glycerol and 0.1 mM MnSO4). For biofilm colony, 3 l of the Bacillus stationary phase cultures was applied to LBGM solid medium containing 1.5% Bacto agar. Salmonella and other pathogens were washed twice with sterile distilled water (SDW) by centrifugation at 2,700 g for 10 minutes, and the pellet was resuspended in SDW. Pathogens (10⁸ cells/ml) were spread on Nutrient agar, then an agar slab of 5 mm in diameter was cut from 3-day bacillus colony and placed on the pathogen layer. Following a 24 hour incubation at 25° C., the plates were checked for inhibition zones. The results in the tables are presented as the mean diameter of the inhibition zone+SD for 3 independent experiments. Images of the colonies on the plates were taken using a 5 megapixel CMOS camera AxioCam ERc 5 s.

### Results

Effect of Growth Media on Salmonella Inhibition

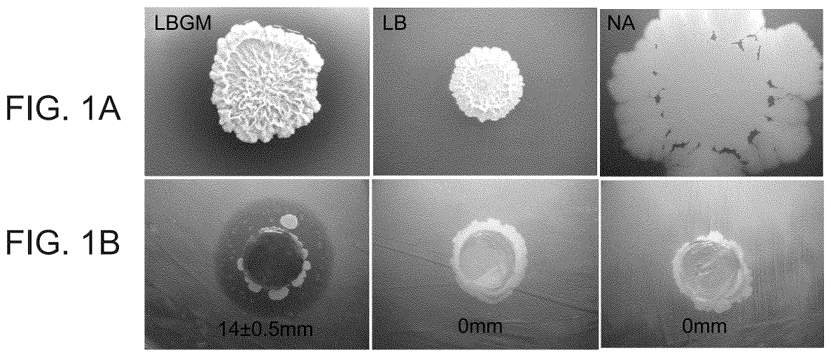

Bacillus subtilis 3610 colony was grown on different agar media for 72 hours and then Salmonella inhibition was estimated (FIGS. 1A-B). Results demonstrate that only colonies grown on LBGM notably inhibit Salmonella growth (14±0.5 mm). Essentially, no Salmonella inhibition was detected for Bacillus colonies developed on LB or NA.

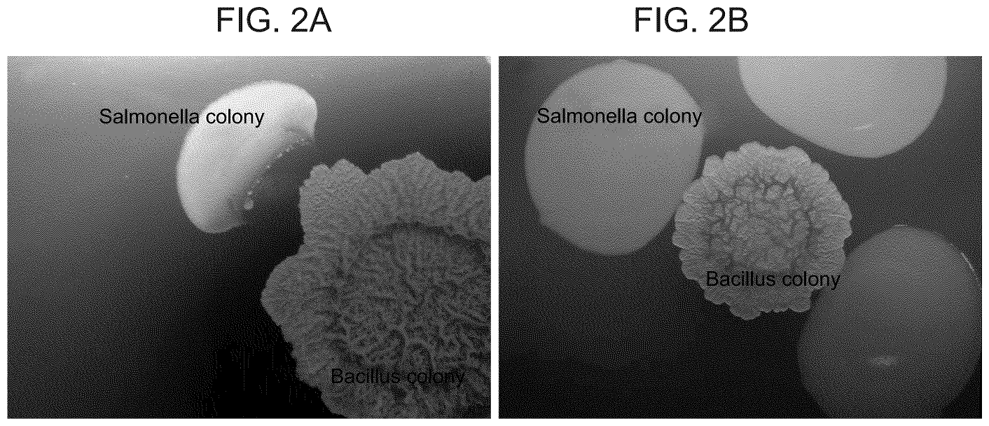

It was further noted that growth inhibition of Salmonella colonies nearby a Bacillus colony occurred on LBGM but not on LB (FIGS. 2A-B). The Salmonella colony was ‘injured’ near the Bacillus colony at the boundary of the pigment secretion.

Kinetics of Bacillus subtilis 3610 Colony Biofilm Formation and Salmonella Inhibition

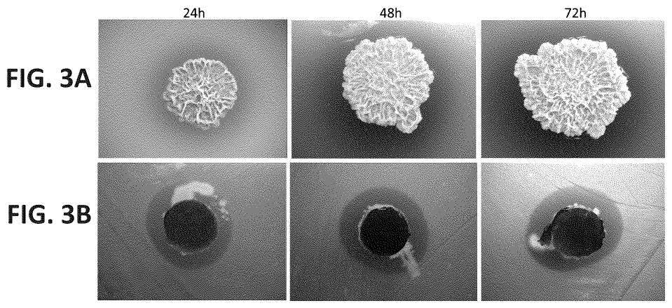

In order to ascertain whether the stage of biofilm formation and accordingly pigment production affects Salmonella inhibition, cells of B. subtilis were grown on LBGM agar for 24, 48 and 72 hours (FIG. 3A). Although more biofilm was formed and pigment secreted with time (FIG. 3A), the level of Salmonella inhibition was not affected significantly at different time points (FIG. 3B).

Biofilm Formation and Salmonella Inhibition

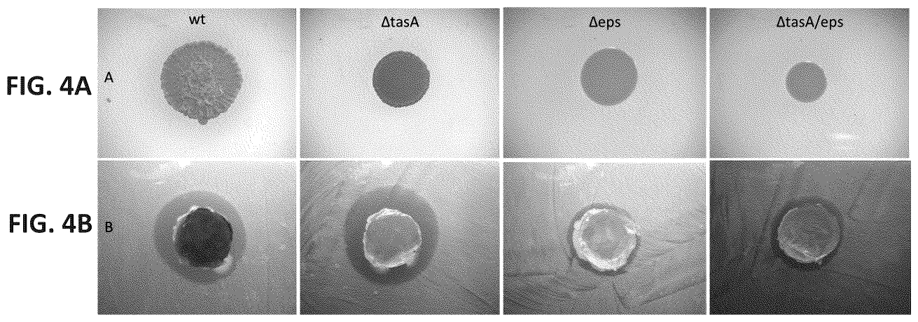

To establish correlation between biofilm formation on LBGM and Salmonella inhibition, two mutants of biofilm formation and double mutant was tested in similar experiments described above. FIG. 4A demonstrates that the tasA mutant partially attenuated biofilm formation whilst the eps and double mutant showed a reduced amount of biofilm formation and pigment secretion. Correlatively, biofilm mutants grown on LBGM, demonstrated different levels of the Salmonella inhibition (FIG. 4B). While, the eps and double mutant demonstrated reduced inhibition of Salmonella compared to WT, the tasA mutant showed a similar inhibition to WT.

Bacillus Colony had Broad-Spectrum Antimicrobial Effect

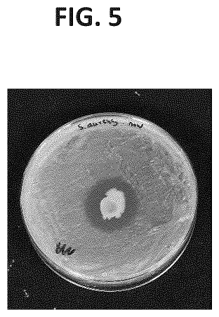

To elucidate whether an antimicrobial pigment would demonstrate a broad-spectrum antimicrobial activity, its inhibitory effect was tested on two Gram-positive and three Gram-negative pathogens using a similar methodology. As can be seen in Table 2, all of the tested species were found to be sensitive to the pigment. Interestingly, the bacterium Staphylococcus aureus was found to be most sensitive to the pigment's activity (FIG. 5).

TABLE 2

| Pathogen | Inhibition diameter (mm) |
|---|---|
| Salmonella Enterica | 13 ± 0.5 |
| Enteropathogenic Escherichia | 12 ± 0.8 |
| coli O55:H7 | |
| Staphylococcus aureus | 20 ± 1 |
| Staphylococcus epidermidis | 18 ± 0.5 |
| Bacillus cereus | 12 ± 0.5 |

### Example 2

Antimicrobial Activity of the Purified Pigment Against Human Pathogens

### Materials and Methods

Isolation of inhibitory pigments: Following removal of Biofilm colonies formed by Bacillus subtilis on a LBGM plate for 3 days at a temperature of 30° C., the agar substrate was collected for subsequent purification. The agar was crushed with a homogenizer in the presence of 25 ml DDW and centrifuged at a speed of 8000 rpm for 10 minutes. The supernatant with the soluble pigment was passed through 0.22-micron filter, frozen and dried in a lyophilizer for 3 days. The resulting precipitate was 10 fold concentrated by re-suspending with 2.5 ml DDW. A black pigment was purified from the Bacillus isolate BB124, a plant derived bacterium that was identified by 16 S ribosomal RNA analysis as B. subtilis while the brown pigment was purified from B. subtilis NCIB 3610.

Preparation of bacterial cultures: Salmonella enterica serovar Typhimurium, Bacillus cereus ATCC 10987 and Staphylococcus aureus ATCC 25923 were used in the experiments. Bacterial cells were maintained as glycerol stocks and stored at −80° C. For each experiment, a fresh culture was made and bacteria were grown in LB broth for 18-20 h at 37° C. with shaking (150 rpm) to obtain stationary phase cultures. Cultures were washed twice with sterile distilled water (SDDW) by centrifugation at 2,700 g for 10 minutes, and the pellet was resuspended in SDDW.

Analysis of anti-microbial activity of purified pigment by agar-diffusion assay: Washed bacterial cells were spread on NA (nutrient agar) plate in a concentration of about 10⁸ CFU/ml. A 5 mm-well was created in the center of the plate using sterile pipette and filled with the purified pigment. A Zone of inhibition around the well was determined after 24 h growth in 25° C.

Analysis of anti-microbial activity of purified pigment in bacterial suspension: For each bacterial strain, the 10⁴ CFU/ml of the cells were grown in LB supplemented by the purified pigment 1:100 (v/v). The tubes were incubated for 24 hours at 25° C. Next, the cell number (CFU) of the pathogens was determined by plating on LB agar plates for Bacillus cereus and Staphylococcus aureus and on XLD plates for Salmonella. Similar experiments were performed in nutrient broth (NB) media for Salmonella only.

### Results

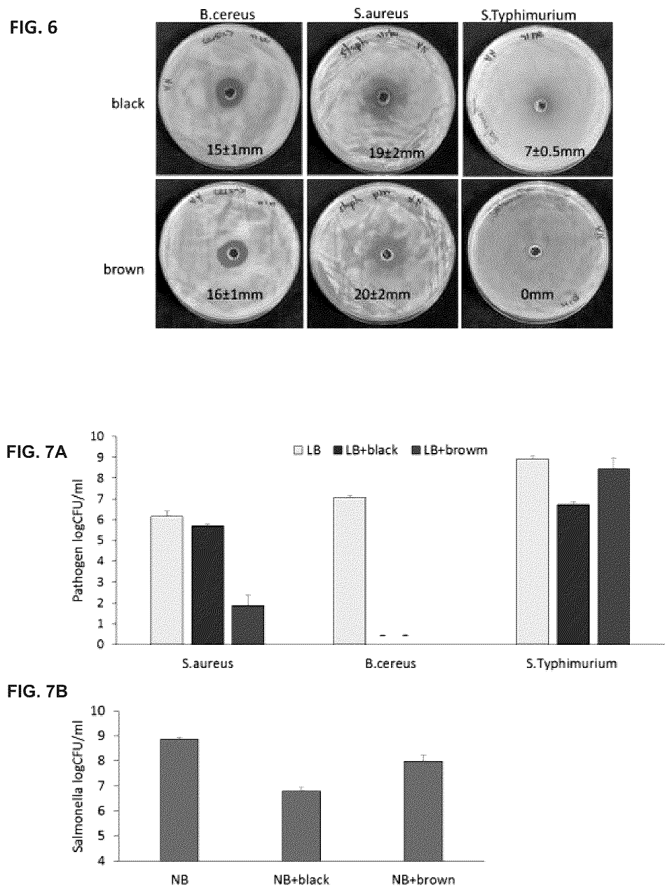

Substantial antimicrobial activity was detected by the black and the brown pigments on the growth of B. cereus and S. aureus on plates. The black and brown pigments demonstrated a similar effect on these two pathogens. In contrast, only slight effect of the black pigment was detected for Salmonella inhibition on a plate (FIG. 6).

On the other hand, strongest effect was detected in liquid for the B. cereus, which was eliminated by the brown or black pigments. For the S. aureus, the brown pigment showed better activity and reduced the bacterial number in four logs compared to the growth in LB. However, the black pigment had stronger effect on Salmonella growth inhibition (also in NB medium) then the brown pigment, and inhibited Salmonella growth by two logs (FIGS. 7A-B).

### Example 3

Sprouts-Derived Medium Induces Anti-Salmonella Activity of Bacillus

### Materials and Methods

Preparation of the sprouts-derived medium: Seeds of alfalfa plant (Medicago sativa) were grown in plastic cups at 25° C. for four days. After germination, 2 grams of sprouts were added to 20 ml of DDW and crushed in Stomacher for 2 cycles of 2 min. The crude extract was centrifuged for 10 min in 6000 rpm and the supernatant was boiled to release the components from the plant derivate. After boiling, the extract was filtered through a 0.22-micron filter.

Bacteria preparation: Bacterial cultures were maintained as glycerol stocks and stored at −80° C. For each experiment, a fresh culture was made and bacteria were grown in LB broth for 18-20 h at 37° C. with shaking (150 rpm) to obtain stationary phase cultures. Salmonella and Bacillus cells were washed twice with sterile distilled water (SDW) by centrifugation at 2,700 g for 10 minutes, and the pellet was resuspended in SDW. Two initial concentration of Salmonella and Bacillus 10² cells/ml or 10⁷ cells/ml were used.

Bacterial strains: Salmonella enterica serovar Typhimurium and Bacillus strain (referred to herein as BX77) was used.

### Results

Salmonella—Bacillus interactions in different growth media

For each experiment, Salmonella and Bacillus (BX77) were added to 12 ml plastic tubes with 1 ml boiled and filtrated sprouts derived medium (BFSM). To determine Salmonella growth in BFSA media without Bacillus, some of the tubes were inoculated with Salmonella only. Tubes were kept in a 25° C. incubator for a maximum of 72 hours. Following 24, 48, 72 hours, Salmonella cell numbers were determined using a colony-forming unit (CFU) plating and counting method on Salmonella selective XLD agar plates. Similar experiments were carried out in Nutrient broth (NB) media.

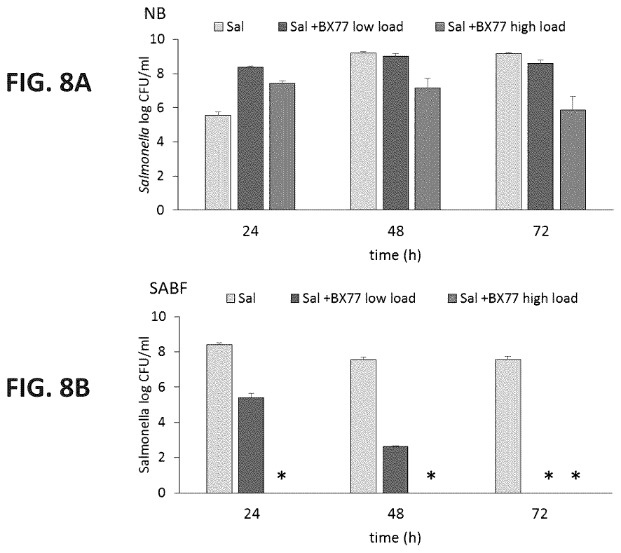

Results summarized in FIGS. 8A-B demonstrate strong Salmonella inhibition and killing following 72 h incubation with Bacillus BX77 grown in BFSA. No Salmonella was detected following 72 h incubation both with low and high Bacillus load.

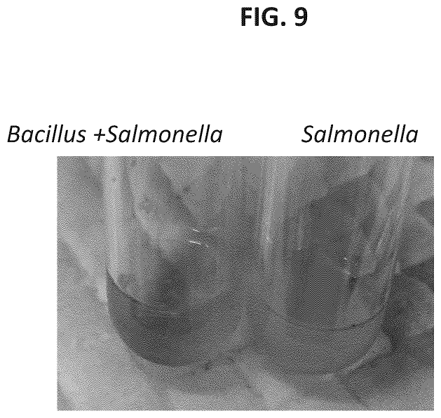

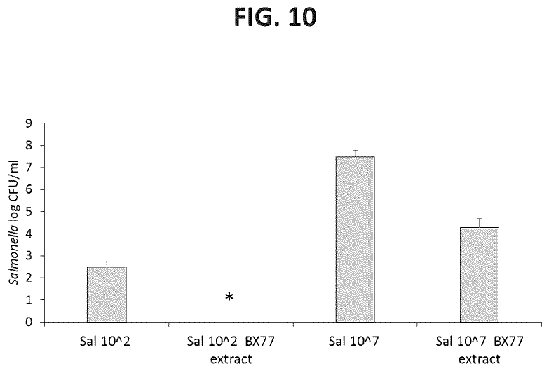

In case of NB, only slight inhibition of Salmonella was detected following the 72 h incubation of Salmonella with high load of Bacillus cells. Interestingly, growth of Bacillus cells resulted in a significant change of the suspension color from white to yellow (FIG. 9). In contrast, Salmonella did not cause a similar change in suspension color. It was therefore hypothesized that yellow pigment secretion by Bacillus cells in sprouts-derived media is related to its anti-Salmonella properties.

### Survival of Salmonella in Conditioned-BFSA Following Incubation with BX77 Cells

To determine whether Bacillus cells secrete anti-bacterial agents when cultured in the presence of BFSA, the medium was inoculated with Bacillus cells for 3 days as described herein above. Following 3 days, BFSA was filtered to remove the Bacillus cells and Salmonella was added for another 24 hours at 25° C., after which the number of Salmonella was determined.

The medium that was inoculated with Bacillus cells inhibited the growth or killed the Salmonella cells, depending on the incubation times. The quantity of the Salmonella cells was reduced by three log when initially inoculated as a high load, whereas it was completely eliminated in the low-cell load. Thus, the results indicate that Bacillus secretes active components against Salmonella into a sprouts-derived media.

### Example 4

### Anti-Salmonella Activity of Bacillus on Alfalfa Seeds

First, Alfalfa (Mledicago sativa) seeds were inoculated with the mCherry-tagged Salmonella in the presence of the GFP-labeled Bacillus cells in dip water for 4 days. Subsequently, fluorescently tagged cells of Salmonella and B. subtilis (strain 3610) were visualized on the alfalfa root (generated through seed germination and the root regrowth) by confocal laser scanning microscope (Olympus IX81, Tokyo, Japan). Red florescence of the Salmonella cells was visualized using excitation wavelength of 543 nm and a BA560-600 nm emission filter, while the green florescence of Bacillus cells were visualized in 488 nm excitation wavelength with BA505-525 nm emission filter. Transmitted light images were obtained using Nomarski differential interference contrast (DIC).

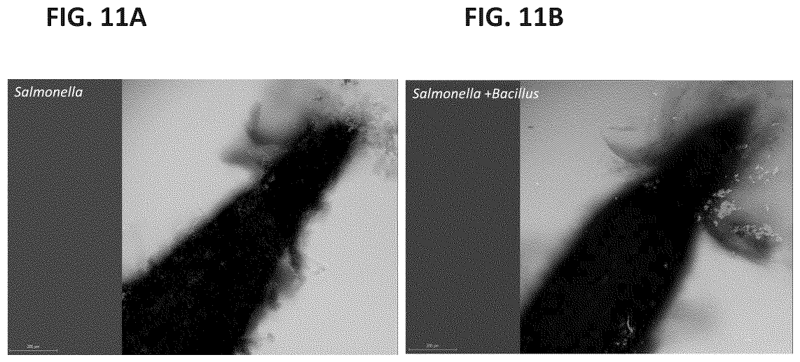

According to the confocal images demonstrated in FIGS. 11A-B, a massive reduction in Salmonella cells was detected on the root in the presence of Bacillus cells. This result indicates that the colonization of the root by Bacillus cells and subsequent biofilm formation can trigger the anti-microbial activity against Salmonella.

### Example 5

### Growth of BX77 in Medicago sativa Extract Increase BX77 Antimicrobial Activity

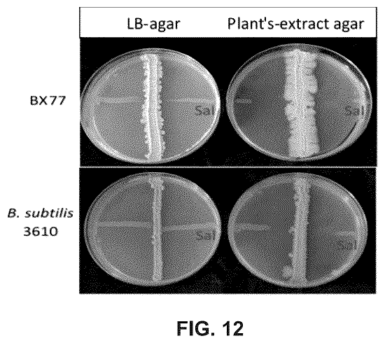

Since BX77 displays antagonistic activity when grown in seedlings of Medicago sativa plants, the effect of the plant's extract to facilitate antimicrobial activity of the bacteria was tested. Antimicrobial activity of BX77 was tested on agar plate prepared with a sterile 4 day old seedling extract, supplemented with 1.5% agar. The extract was prepared by crushing the seedlings (1 g in 9 ml sterile water) in a stomacher for 2 minutes and removal of suspended particles by centrifugation and filtration through a 0.22 μm membrane. The final extract was sterilized by autoclaving. The anti-microbial activity of BX77 and a laboratory B. subtilis 3610 strain was tested in a rich medium (L_B) and in the plant extract agar against Salmonella folloxwing 24 h, at 25° C. (FIG. 12). Both strains inhibited the growth of Salmonella; however, the growth of BX77 and its inhibition zone was much higher than that of the B. subtilis 3610 strain. These findings indicate that the Medicago sativa extract contains factor(s) that enhance the growth of BX77 and/or induce antibacterial activity thereof.

### Example 6

### Effect of Antimicrobials Secreted During Growth of BX77 on Salmonella

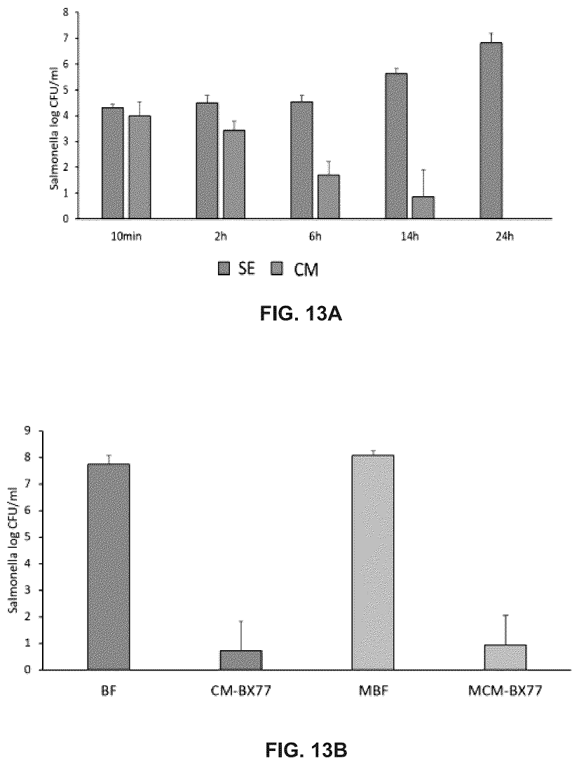

In order to study the effect of the antimicrobials secreted by BX77 on Salmonella. BX77 was grown in Medicago sativa plant extract for 72 h at 25° C. and conditioned medium (CM) was prepared from the culture medium. Briefly, bacteria were removed by centrifugation and filtered and the medium was heat-sterilized. Salmonella (10⁴ CFU/ml) was added to the CM or to a sterile Medicago sativa plant extract and incubated for 24 h, at 24° C. The concentration of Salmonella steadily increased with time in the plant's extract, while in the presence of CM, the concentration of Salmonella steadily decreased (FIG. 13A).

To test if similar factors exist in additional plants, a plant extract from 4-days old Vigna radiate seedlings was prepared. Salmonella (10⁴ CFU/nil) was added to a sterile plant extract, with or without BX77 (10⁷ CFU/ml) and incubated for 24 h at 25° C. The effect of both M. sative and V. radiate extracts, and their respective conditioned media had a similar effect on Salmonella (FIG. 13B), suggesting that extracts from both plants induce the secretion of antimicrobial agents.

Treatment of the M. sative CM prepared following incubation of BX77 for 24 h at 25° C., with proteinase K did not affect the activity of the CM (data not shown), suggesting that the antimicrobial(s) is resistant to protein degradation. The heat-sterilization of the CM suggest that the active compounds are resistant to heat (up to 121° C.).

### Example 7

### Dynamics of BX77 Sporulation and Secretion of Antimicrobial Metabolites

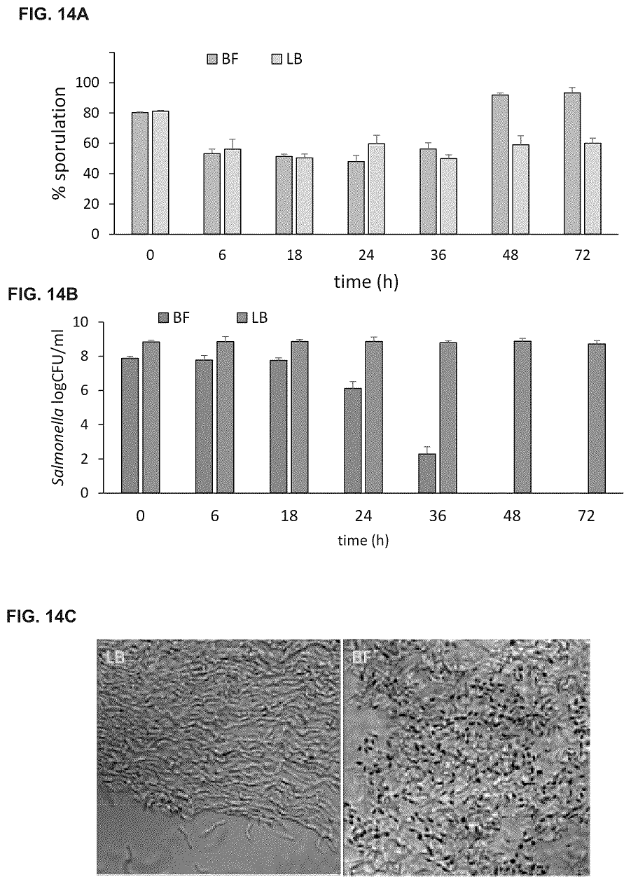

In order to study the kinetics of the generation and secretion of the antimicrobial agent, BX77 was grown for 72 h at 25° C. in M. sative extract and samples from the culture were collected at different time points. Each sample was tested for the presence of sporulated bacteria and used to prepare a conditioned medium (CM). The antimicrobial activity of each CM sample was also tested following addition of Salmonella (10⁴ CFU/ml), and incubation for another 24 hours at 25° C. The number of endospores and the effect of CM, derived from different growth times, on Salmonella counts are present in FIG. 14A and FIG. 14B. Substantial activity of antimicrobials appeared at 24 h and maximal activity was demonstrated at 48 and 72 h. A representative microscopic image showing a high abundance of endospores at 48 h, in the plant-extract culture, is presented in FIG. 14C.

### Example 8

### BX77 has a Broad-Rage Antimicrobial Activity

The activity of BX77 strain was tested on agar plates against several bacterial and fungal pathogens as well as against human pathogens and spoilage bacteria (FIG. 15). BX77 strain was found to be highly active against the tested bacterial and fungal phytopathogens. Aspergillus flavus Fusarium proliferatu and Pencillium expansum.

BX77 was also active against the food/beverages-spoilage bacterium Alicyclobacillus acidoterrestris as well as against the foodborne pathogen, Bacillus cereus. In contrast, BX77 was inactive against Pseudomonas aeruginosa.

BX-77 was also found to inhibit the growth of Pseudomonas syringae, Pectobacterium carotovorum and Clavibacter michiganensis, three plant pathogens. BX-77 highly inhibited also the growth of Bacillus cereus, Listeria innocua, Pseudomonas fluorescence and Alicyclobacillus acidiphilus.

The results are summarized in Table 3, herein below.

TABLE 3

| | | Inhibition zone |
|---|---|---|
| Bacterial species | Origin | (mm) |
| S. Typhimurium (SL1344) | Human pathogen | 15 ± 2 |
| E. coli O55:H7 | Human pathogen | 15 ± 2 |
| Pseudomonas aeruginosa | Human | — |
| Staphylococcus aureus | Human pathogen | 19 ± 2 |
| Bacillus cereus | Human pathogen | 22 ± 2 |
| Listeria innocua | Environment | 14 ± 2 |
| Pseudomonas fluoresence | Environment | 25 ± 2 |
| Alicyclobacillus acidiphilus | Spoilage | 22 ± 2 |
| | bacterium | |
| Pseudomonas syringae | Plant pathogen | 18 ± 3 |
| Pectobacterium carotovorum | Plant pathogen | 15 ± 2 |
| Clavibacter michiganensis | Plant pathogen | 24 ± 3 |

### Example 9

### Bacillus BX77 Inhibits the Colonization of Salmonella on Germinated Medicago sativa Seeds

To investigate if BX77 can be used as a biocontrol agent to inhibit growth of Salmonella on plants, Medicago sativa seeds were artificially contaminated with a low inoculum of S. enterica serovar Typhimurium (10⁶ CFU/g) expressing the fluorescent mCherry protein and the seeds placed in a mini-sprouter containing sterile water enriched with BX77 cells (10⁷ CFU/ml) for 4 days at 25° C. Seeds germinated on BX77-free water served as a control. The presence of Salmonella on the young seedlings at 4 days was examined by confocal microscopy (FIG. 16). Non-treated seeds show the Salmonella cells (red fluorescence) in various regions of the seedling, while in BX77-treated seeds, no fluorescence was observed suggesting that BX77 inhibits Salmonella colonization on the growing seedlings.

### Example 10

### BX77 Expresses Known Genes Associated with Antimicrobial Activity at 48 h but not at 24 h

Potential expression of selected antimicrobial agents in BX77 was tested by PCR analysis of known genes involved in the synthesis of antimicrobial agents in Bacillus. The details of the genes and primer sequences are set forth in Table 4.

TABLE 4

| Gene | Antimicrobial | Sequence |
|---|---|---|
| fen | Fengycin | Fwd:-5′-CCGCAGCAGGTCGTATTAAA-3′ (SEQ ID |
| | | NO: 4) |
| | | Rev:-5′-GGTCTCTTCGTTTGGCTTCT-3 (SEQ ID NO: |
| | | 5) |
| diffD | Difficidin | Fwd:-5′-CGT GTG CCG AAG GAG AAA TA-3′ (SEQ |
| | | ID NO: 6) |
| | | REV:-5′-CGA ATC GTA CAG ACG GGA AAT-3′ |
| | | (SEQ ID NO: 7) |
| macL | Macrolactin | FWD:-5′-GCG GGC ATT ACG AAA GTT ATT C-3′ |
| | | (SEQ ID NO: 8) |
| | | REV:-5′-GGC ACT GTC CGG TAC ATA AA-3 (SEQ |
| | | ID NO: 9) |
| bacL | Bacillaene | FWD CGA TGG CCA ATG CGT TTA TG (SEQ ID NO: |
| | | 10) |
| | | REV ATG CAG CCG TCT TTC CAA (SEQ ID NO: 11) |
| butN | Butracin | FWD 5′-CAG GTG AAG CTG TTT GGA ATG-3′ |
| | | (SEQ ID NO: 12) |
| | | REV 5′-CAG GCG ATT TGA CCG TAT CT-3′ (SEQ |
| | | ID NO: 13) |
| srfA | Surfactin | FWD 5′-CGGTATGAGTCGATGAGAATCC-3′ (SEQ |
| | | ID NO: 14) |
| | | REV 5′-CTGATATCTGTGTGCCCGATAG-3′ (SEQ ID |
| | | NO: 15) |
| bacE | Bacilysin | Fwd 5′-TTCTCCCGAAAGGCGAATATC-3′ (SEQ ID |
| | | NO: 16) |
| | | Rev 5′-CCGCTTCTAAAGACGACATGA-3 (SEQ ID |
| | | NO: 17) |
| bmyA | Bacillomycin | Fwd 5′-GAATTCTGGCGGCTAAGG-3′ (SEQ ID NO: |
| | D | 18) |
| | | Rev 5′-GGAAGGAAGTGCTCGGAATAA-3 (SEQ ID |
| | | NO: 19) |

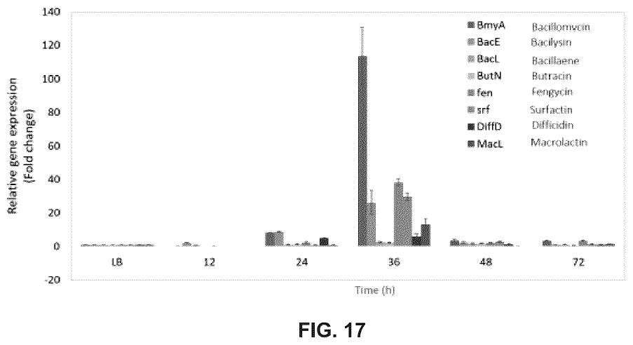

qPCR was performed on mRNA templates derived from BX77 culture grown in LB or in plant-extract for up to 72 h at 25° C. Gene expression is presented as a relative expression where expression in LB broth is considered 1.0 (FIG. 17). The expression of the bmyA, bacE and diifD genes increase at 24 h and continue till 36 h where bmyA is expressed 100-folds higher than in LB-medium. A high expression is seen also in the bacE, fen, srf and macL genes, while expression of the diffD remain at the same level as at 24 h. Interestingly, the expression of all these genes decrease at 48 and 72 h, where BX77 expresses its maximum antimicrobial activity (FIG. 17).

### Example 11

### Materials and Methods

Seed sterilization by Ca-hypochlorite: To assess the ability of Ca-hypochlorite to inactivate the Salmonella enterica serovar Typhimurium (STm) strain SL1344 on the seeds, alfalfa seeds were pre-contaminated with increasing concentrations of STm cells. Breifly, seeds (1 g) 15 were contaminated with the desired number of bacteria by adding 20 μL of STm suspended in SDDW and mixing by a sterile plastic spreader and then the seeds were dried at room-temp for 30 minutes. Contaminated seeds were disinfected by soaking the seeds in 5 ml of 20,000 ppm Ca-hypochlorite (Lonza, Arch Chemicals, Inc., Norwalk, CT) for 30 minutes, while occasionally hand shaking the tubes. The seeds were washed 5 times with 10 ml sterile tap water while mixing, then spread over the Whatman paper in the inner cup of the sprouter and were grown as described above. After 4 days the alfalfa sprouts were extracted and the STm concentration in the extract was analyzed. Salmonella Typhimurium SL1344 survival in sprouts in the presence or absence of Bacillus BX-77 was analyzed after disinfection by Ca-hypochlorite of alfalfa seeds contaminated by 10² or 10⁵ CFU of STm.

### Results

Efficiency of Decontamination of Salmonella on Alfalfa Seeds by Ca-Hypochlorite

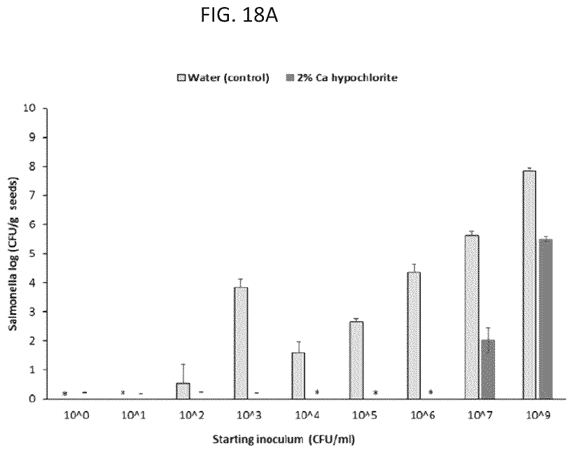

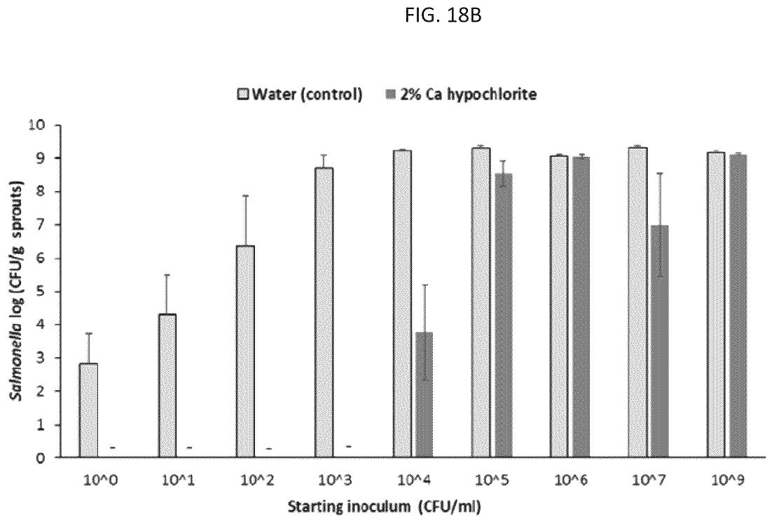

Alfalfa seeds were contaminated with Salmonella by dipping in 10²-109 CFU/ml. The efficiency of decontamination by Ca-hypochlorite and the influence on the concentration of Salmonella was analyzed. The number of Salmonella on the seeds (CFU/g seeds) was detected immediately after the contamination and in the sprouts after 3 days of sprouting (FIGS. 18A-B).

The decontamination of the seeds was efficient after infection by low Salmonella concentrations up to 10³/ml and Salmonella was not detected even after sprouting. On the other hand when the concentrations were higher, the decontamination was not efficient as detected in also in 3 day sprouts.

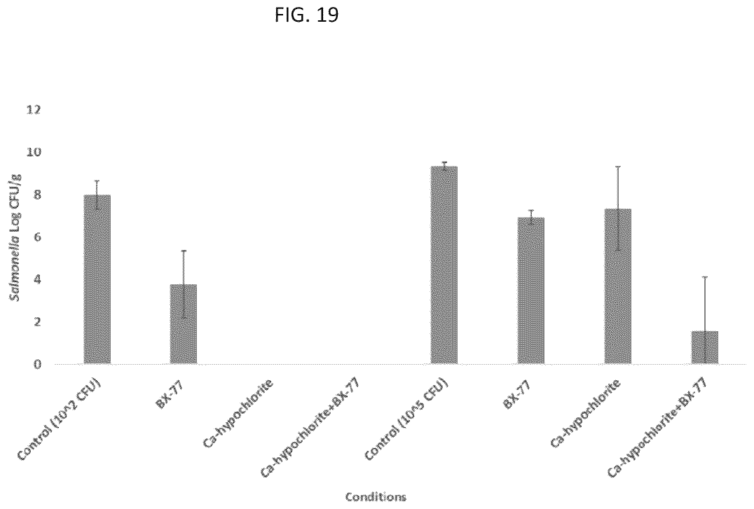

Alfalfa seeds were infected by 10² or 10⁵ CFU of Salmonella, dried, disinfected by Ca-hypochlorite and seeded in the presence or absence of BX-77. Salmonella infected seeds were used as controls in the absence or the presence of BX-77. As illustrated in FIG. 19, the salmonella growth of the control samples on the left set increased from 2 logs CFU/g to about 8 log CFU/g. Addition of BX-77 reduced the growth by 4.2 log CFU/g. Disinfection with Ca-hypochlorite with or without BX-77 killed all the Salmonella cells. The Salmonella growth of the control samples on the right set increased from 5 logs CFU/g to about 9.3 log CFU/g. Addition of BX-77 reduced the growth by 2.4 log CFU/g only. Disinfection with Ca-hypochlorite reduced the growth by 2 log CFU/g and disinfection with Ca-hypochlorite and treatment with BX-77, resulted in reduction of the Salmonella cells by 7.8 logs CFU/g. With high concentration of salmonella, BX-77 or Ca-hypochlorite had low effect on killing salmonella, but together there was synergistic effect and they caused reduction of salmonella by about 8 logs CFU/g. Sprouts from seeds that were treated with Ca-hypochlorite and sprouted, with or without BX-77 also grew better.

### Example 12

### Anti-E. coli Activity in Sprouted Alfalfa Seeds

Alfalfa seeds contaminated with E. coli 055:H7 (100 CFU/g) were sprouted as described above in the presence or absence of individual Bacillus strains at a concentration of 10⁷ CFU/ml. In the absence of Bacillus strain, the E. coli reached about 4 log CFU/g after 4 days of sprouting, compared to Salmonella, which reached 8 log CFU/g. BX-77 strain completely inhibited the growth of E. coli.

### Example 13

BX-77 Strain is Active Against Multiple Salmonella Serovars

Growth inhibition activity of BX-77 was measured against Salmonella serovars other than Typhimurium (S. Infantis, S. Enteritidis, S. Virchow and S. Hadar), using the zone inhibition assay as well as in sprouted seeds. In both methods, BX-77 had growth inhibition activity against the serovars analysed.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of generating an antimicrobial agent comprising:
(a) culturing Bacillus cells of the species subtilis or velezensis in a medium under conditions effective to allow secretion of at least one antimicrobial agent into the medium; and
(b) purifying said at least one antimicrobial agent from the medium to generate a purified preparation comprising said at least one antimicrobial agent, thereby generating the antimicrobial agent.

2. The method of claim 1, further comprising testing the activity of the antimicrobial agent.

3. The method of claim 1, wherein said Bacillus cells are of the species subtilis.

4. The method of claim 3, wherein said conditions support generation of a biofilm of said Bacillus subtilis cells or enhance secretion of a pigment secreted from said Bacillus subtilis cells.

5. The method of claim 1, wherein said purifying is effected by size exclusion or by reverse phase column chromatography.

6. The method of claim 1, wherein said antimicrobial agent is an antibacterial agent which is bactericidal towards at least one of the bacteria selected from the group consisting of E. coli, Salmonella Enterica, Staphylococcus aureus, Staphylococcus epidermidis and Bacillus cereus.

7. The method of claim 1, wherein said Bacillus velezensis has a 16S rRNA sequence as set forth in SEQ ID NO: 1.

8. A composition of matter comprising at least one antimicrobial agent generated according to the method of claim 1.

9. A composition of matter comprising at least one antimicrobial agent secreted from a Bacillus bacteria of the species subtilis or velezensis, wherein said at least one antimicrobial agent is more than 1% of the Bacillus components in the composition of matter.

10. A method of treating an infection in a subject in need thereof, the method comprising administering to the subject a therapeutically effective amount of the composition of generated according to the method of claim 1, thereby treating the infection.

11. The method of claim 10, wherein the infection is a bacterial infection.

12. The method of claim 10, wherein the bacterial infection is selected from the group consisting of an E. coli infection, a Salmonella Enterica infection, a Staphylococcus aureus infection, a Staphylococcus epidermidis infection and a Bacillus cereus infection.

13. A method of treating an infection in a subject in need thereof, the method comprising administering to the subject a therapeutically effective amount of the composition of claim 9, thereby treating the infection.

14. The method of claim 13, wherein the infection is a bacterial infection.

15. The method of claim 13, wherein the bacterial infection is selected from the group consisting of an E. coli infection, a Salmonella Enterica infection, a Staphylococcus aureus infection, a Staphylococcus epidermidis infection and a Bacillus cereus infection.

16. A bacterial culture comprising Bacillus bacteria of the species subtilis or velezensis and a heterologous plant which promotes antimicrobial activity of said Bacillus bacteria.

17. The bacterial culture of claim 16, wherein said plant is a sprout.

18. The bacterial culture of claim 17 wherein said sprout is a Medicago sativa sprout or a Vigna radiate sprout.

19. A method for prolonging the shelf life of a plant or the post-harvest quality of a plant, or a combination thereof comprising post-harvest contacting said plant with an effective amount of Bacillus bacteria of the species subtilis or velezensis, thereby prolonging the shelf life of a plant or the post-harvest quality of a plant, or a combination thereof.

20. The method of claim 19, wherein said Bacillus bacteria has a 16S rRNA sequence as set forth in SEQ ID NO: 1.
